(19) United States
(12) Patent Application Publication
(10) Pub. No.: US 2007/0275967 A1
(43) Pub. Date: Nov. 29, 2007

(54) Pyrazolinols

(75) Inventors: Jürgen Scherkenbeck, Wermelskirchen (DE); Jutta Bohmer, Bracknell (GB); Klaus Kunz, Dusseldorf (DE); Oliver Gaertzen, Koln (DE); Ulrich Gorgens, Ratingen (DE); Peter Losel, Leverkusen (DE); Olga Malsam, Rosrath (DE); Udo Reckmann, Koln (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: BAYER CROPSCIENCE AKTIENGESELLSCHAFT, Monheim (DE)

(21) Appl. No.: 10/583,310

(22) PCT Filed: Dec. 7, 2004
(86) PCT No.: PCT/EP04/13875

§ 371 Date: Jul. 9, 2007

(30) Foreign Application Priority Data
Dec. 17, 2003 (DE) 103 59 074.9

(51) Int. Cl. A01N 43/56; A01N 43/40; A01N 43/60; A01N 43/84; A01P 15/00; C07D 231/08; C07D 401/12; C07D 403/12; C07D 413/12

(52) U.S. Cl. 514/236.5; 514/254.05; 514/341; 514/403; 544/140; 544/371; 546/276.1; 548/369.4

(57) ABSTRACT

The use of pyrazolinols formula (I) 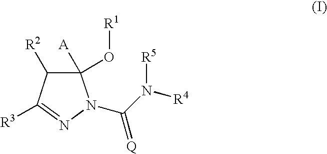
in which A, Q, R¹, R², R³, R⁴ and R⁵ are as defined in the description for controlling pests, novel pyrazolinols and a plurality of processes for preparing these compounds.

The present application of an invention relates to the use of known and novel pyrazolinols and derivatives thereof as crop treatment agents, in particular for the control of animal pests, to crop treatment agents based on known and novel pyrazolinols and derivatives thereof, and to novel trifluoromethylpyrazolinols and derivatives thereof, and also to processes for their preparation.

A relatively large number of pyrazolinol derivatives is already known from the literature [cf. Chemistry of Heterocycl. Compounds (Translation of Khim. Geterots. Soedin.) 35 (1999), 87-92; Doklad. Akad. Nauk Respub. Uzbekistan 2000, 4245—cited in Chem. Abstracts 136:69766; Eur. J. Pharmacol. 451 (2002), 141-147; Heteroatom Chemistry 14 (2003), 132-137; J. Chem. Res., Synopses 2001, 12-13, 133-147; J. Fluorine Chem. 92 (1998), 23-26; ibid. 99 (1999), 177-182; J. Med. Chem. 14 (1971), 997-998; Khim. Geterots. Soedin. 1986, 128—cited in Chem. Abstracts 105:172346; Khim. Geterots. Soedin. 1990, 1260-1270—cited in Chem. Abstracts 114:228877; Khim. Geterots. Soedin. 1993, 267-268—cited in Chem. Abstracts 120:54516; Russian J. Org. Chem. (Translation of Zhurnal Organi. Khim.) 35 (1999), 357-363; ibid. 36 (2000), 1144-1149; Spectroscopy Letters 32 (1999), 851-865; Synthesis 2002; 1679-1083; ibid. 2002, 2353-2358; Synthetic Commun. 30 (2000), 1457-1465; Tetrahedron 55 (1999), 345-352; Tetrahedron Lett. 43 (2002), 8701-8705; Zhurnal Obshchei Khimii 57 (1987), 584-595—cited in Chem. Abstracts 108:150362; Zhurnal Organi. Khimii 31 (1995), 937-943—cited in Chem. Abstracts 124:260918].

However, these compounds have only been described as products of chemical scientific research and without reference to any commercial use.

It has now been found that the pyrazolinols, some of which are known, and their derivatives of the formula (I) 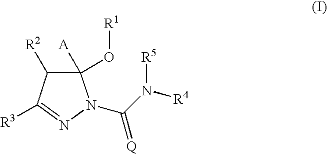
in which
A represents optionally substituted alkyl,
Q represents oxygen or sulfur,
R¹ represents hydrogen or represents in each case optionally substituted alkyl, alkylcarbonyl, alkenyl, alkenylcarbonyl, cycloalkyl, cycloalkylcarbonyl, aryl, arylcarbonyl, arylalkyl or arylalkylcarbonyl,
R² represents hydrogen or represents in each case optionally substituted alkyl, alkylcarbonyl, alkoxycarbonyl, cycloalkyl, aryl, arylalkyl or heterocyclyl,
R³ represents in each case optionally substituted alkyl, alkenyl, cycloalkyl, aryl, arylalkyl or heterocyclyl,
R² and R³ furthermore together represent alkanediyl(alkylene),
R⁴ represents hydrogen, represents amino, or represents in each case optionally substituted alkyl, alkylcarbonyl, alkylsulfonyl, alkylamino, alkylcarbonylamino, alkylsulfonylamino, alkenyl, alkenylcarbonyl or alkynyl, and
R⁵ represents hydrogen or represents in each case optionally substituted alkyl, alkenyl, alkynyl, cycloalkyl, cycloalkylalkyl, aryl, arylalkyl, heterocyclyl or heterocyclylalkyl,
   are highly active against animal pests.

Saturated or unsaturated hydrocarbon radicals, such as alkyl or alkenyl, are in each case straight-chain or branched as far as this is possible—including in combination with heteroatoms, such as in alkoxy.

Optionally substituted radicals can be mono- or polysubstituted, where in the case of polysubstitution the substituents can be identical or different.

The compounds of the general formula (I) according to the invention contain at least one asymmetrically substituted carbon atom and can therefore be present in different enantiomeric (R- and S-configured forms) or diastereomeric forms. The invention relates both to the use of the different possible individual enantiomeric or stereoisomeric forms of the compounds of the general formula (I) and to the mixtures of these isomeric compounds.

Preferred substituents or ranges of the radicals listed in the formulae above and below are described below.
A preferably represents optionally hydroxyl-, cyano-, halogen-, C₁-C₄-alkoxy- or C₁-C₄-haloalkoxy-substituted C₁-C₈-alkyl.
A particularly preferably represents in each case optionally hydroxyl-, cyano-, fluorine-, chlorine-, bromine-, methoxy-, ethoxy-, n- or isopropoxy-, monofluoromethoxy-, difluoromethoxy-, trifluoromethoxy-, monofluoroethoxy-, difluoroethoxy- or trifluoroethoxy-substituted methyl, ethyl, n- or isopropyl, n-, iso- or sec-butyl, pentyl, hexyl, heptyl or octyl.
A very particularly preferably represents in each case optionally fluorine- and/or chlorine-substituted methyl, ethyl, n- or isopropyl, n-, iso- or sec-butyl, hexyl or octyl.
A especially preferably represents difluoromethyl, trifluoromethyl, fluorodichloromethyl, chlorodifluoromethyl, trichloromethyl, 1,1,2,2-tetrafluoroethyl, pentafluoroethyl, heptafluoro-n-propyl, heptafluoroisopropyl, nonafluorobutyl, perfluorohexyl or perfluorooctyl.
A represents, with emphasis, difluoromethyl, trifluoromethyl, fluorodichloromethyl, chlorodifluoromethyl, trichloromethyl, 1,1,2,2-tetrafluoroethyl or pentafluoroethyl, especially trifluoromethyl.
Q preferably represents oxygen.
Q furthermore preferably represents sulfur.
R¹ preferably represents hydrogen, represents in each case optionally hydroxyl-, cyano-, halogen-, C₁-C₄-alkoxy- or C₁-C₄-haloalkoxy-substituted C₁-C₁₀-alkyl or (C₁-C₁₀-alkyl)-carbonyl, represents in each case optionally cyano- or halogen-substituted C₂-C₁₀-alkenyl or (C₂-C₁₀-alkenyl)carbonyl, represents in each case optionally cyano-, halogen-, C₁-C₄-alkyl- or C₁-C₄-haloalkyl-substituted C₃-C₆-cycloalkyl or (C₃-C₆-cycloalkyl)carbonyl, represents in each case optionally nitro-, cyano-, halogen-, C₁-C₄-alkyl-, C₁-C₄-haloalkyl-, C₁-C₄-alkoxy- or C₁-C₄-haloalkoxy-substituted C₆-C₁₀-aryl or (C₆-C₁₀-aryl)carbonyl, or represents in each case optionally nitro-, cyano-, halogen-, C₁-C₄-allyl-, C₁-C₄-haloalkyl-, C₁-C₄-alkoxy- or C₁-C₄-haloalkoxy-substituted (C₆-C₁₀-aryl)-C₁-C₄-alkyl or (C₆-C₁₀-aryl-C₁-C₄-alkyl)carbonyl.
R¹ particularly preferably represents hydrogen, represents in each case optionally cyano-, fluorine-, chlorine-, bromine-, methoxy-, ethoxy-, n- or isopropoxy-, n-, iso-, sec- or tert-butoxy-substituted methyl, ethyl, n- or isopropyl, n-, iso-, sec- or tert-butyl, n-, iso-, sec-, tert- or neo-pentyl, acetyl, propionyl, n- or isobutyroyl, represents in each case optionally cyano-, fluorine-, chlorine- or bromine-substituted propenyl, butenyl, pentenyl, propenylcarbonyl, butenylcarbonyl or pentenylcarbonyl, represents in each case optionally cyano-, fluorine-, chlorine-, bromine-, methyl-, ethyl-, n- or isopropyl-substituted cyclopropyl, cyclobutyl, cyclopentyl, cyclohexyl, cyclopropylcarbonyl, cyclobutylcarbonyl, cyclopentylcarbonyl or cyclohexylcarbonyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted phenyl, naphthyl, phenylcarbonyl or naphthylcarbonyl, or represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted benzyl, phenylethyl, naphthylmethyl, phenylmethylcarbonyl, phenylethylcarbonyl or naphthylmethylcarbonyl.
R¹ very particularly preferably represents hydrogen, represents in each case optionally cyano-, fluorine-, chlorine-, methoxy-, ethoxy-, n- or isopropoxy-substituted methyl, ethyl, n- or isopropyl, n-, iso- or sec-butyl, acetyl, propionyl, n- or isobutyroyl, represents in each case optionally fluorine- or chlorine-substituted propenyl, butenyl, propenylcarbonyl or butenylcarbonyl, represents in each case optionally fluorine-, chlorine- or methyl-substituted cyclopropyl, cyclopentyl, cyclohexyl, cyclopropylcarbonyl, cyclopentylcarbonyl or cyclohexylcarbonyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted phenyl or phenylcarbonyl, or represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted benzyl, phenylethyl, phenylmethylcarbonyl or phenylethylcarbonyl.
R¹ especially preferably represents hydrogen.
R² preferably represents hydrogen, represents optionally cyano-, halogen- or C₁-C₄-alkoxy-substituted C₁-C₁₀-alkyl, (C₁-C₁₀-alkyl)carbonyl or (C₁-C₁₀-alkoxy)carbonyl, represents optionally cyano-, halogen- or C₁-C₄-alkyl-substituted C₃-C₆-cycloalkyl, represents in each case optionally nitro-, cyano-, halogen-, C₁-C₄-alkyl-, C₁-C₄-haloalkyl-, C₁-C₄-alkoxy- or C₁-C₄-haloalkoxy-substituted C₆-C₁₀-aryl or C₆-C₁₀-aryl-C₁-C₄-allyl, or represents optionally nitro-, cyano-, halogen-, C₁-C₄-alkyl-, C₁-C₄-haloalkyl-, C₁-C₄-alkoxy- or C₁-C₄-haloalkoxy-substituted heterocyclyl having up to 6 carbon atoms and at least one heteroatom from the group consisting of N, O, S.
R² particularly preferably represents hydrogen, represents in each case optionally cyano-, fluorine-, chlorine-, bromine-, methoxy-, ethoxy-, n- or isopropoxy-, n-, iso-, sec- or tert-butoxy-substituted methyl, ethyl, n- or isopropyl, n-, iso-, sec- or tert-butyl, acetyl, propionyl, n- or isobutyroyl, methoxycarbonyl, ethoxycarbonyl, n- or isopropoxycarbonyl, represents in each case optionally cyano-, fluorine-, chlorine-, bromine-, methyl-, ethyl-, n- or isopropyl-substituted cyclopropyl, cyclobutyl, cyclopentyl or cyclohexyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluoro-chloroethoxy-, or trifluoroethoxy-substituted phenyl, naphthyl, benzyl or phenylethyl, or represents optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluoro-chloroethoxy- or trifluoroethoxy-substituted heterocyclyl from the group consisting of furyl, tetrahydrofuryl, thienyl, pyrrolyl, pyrazolyl, oxazolyl, isoxazolyl, thiazolyl, isothiazolyl, oxadiazolyl, thiadiazolyl, pyridinyl, pyrazinyl, pyridazinyl, pyrimidinyl.
R² very particularly preferably represents hydrogen, represents in each case optionally cyano-, fluorine-, chlorine-, methoxy-, ethoxy-, n- or isopropoxy-substituted methyl, ethyl, n- or isopropyl, acetyl, propionyl, methoxycarbonyl or ethoxycarbonyl, represents in each case optionally fluorine-, chlorine- or methyl-substituted cyclopropyl, cyclopentyl or cyclohexyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted phenyl, benzyl or phenylethyl, or represents optionally cyano-, fluorine-, chlorine-, bromine-, methyl-, ethyl-, n- or isopropyl-, fluoromethyl-, chloromethyl-, difluoromethyl-, dichloromethyl-, trifluoromethyl-, trichloromethyl-, methoxy-, ethoxy-, n- or isopropoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted heterocyclyl from the group consisting of furyl, thienyl, pyrrolyl, pyrazolyl, oxazolyl, isoxazolyl, thiazolyl, pyridinyl, pyrimidinyl.
R² especially preferably represents hydrogen or methyl.
R³ preferably represents optionally cyano-, halogen- or C₁-C₄-alkoxy-substituted C₁-C₁₀-alkyl, represents optionally cyano-, halogen- or C₁-C₄-alkyl-substituted C₃-C₆-Cycloalkyl, represents in each case optionally nitro-, cyano-, halogen-, C₁-C₄-alkyl-, C₁-C₄-haloalkyl-, C₁-C₄-alkoxy- or C₁-C₄-haloalkoxy-substituted C₆-C₁₀-aryl or C₆-C₁₀-aryl-C₁-C₄-alkyl, or represents optionally nitro-, cyano-, halogen-, C₁-C₄-alkyl-, C₁-C₄-haloalkyl-, C₁-C₄-alkoxy- or C₁-C₄-haloalkoxy-substituted heterocyclyl having up to 6 carbon atoms and at least one heteroatom from the group consisting of N, O, S.
R³ particularly preferably represents in each case optionally cyano-, fluorine-, chlorine-, bromine-, methoxy-, ethoxy-, n- or isopropoxy-, n-, iso-, sec- or tert-butoxy-substituted methyl, ethyl, n- or isopropyl, n-, iso-, sec- or tert-butyl, represents in each case optionally cyano-, fluorine-, chlorine-, bromine-, methyl-, ethyl-, n- or isopropyl-substituted cyclopropyl, cyclobutyl, cyclopentyl or cyclohexyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted phenyl, naphthyl, benzyl or phenylethyl, or represents optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted heterocyclyl from the group consisting of furyl, tetrahydrofuryl, thienyl, pyrrolyl, pyrazolyl, oxazolyl, isoxazolyl, thiazolyl, isothiazolyl, oxadiazolyl, thiadiazolyl, pyridinyl, pyrazinyl, pyridazinyl or pyrimidinyl.
R³ very particularly preferably represents in each case optionally cyano-, fluorine-, chlorine-, methoxy-, ethoxy-, n- or isopropoxy-substituted methyl, ethyl, n- or isopropyl, isobutyl or tert-butyl, represents in each case optionally fluorine-, chlorine- or methyl-substituted cyclopropyl, cyclopentyl or cyclohexyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted phenyl, naphthyl, benzyl or phenylethyl, or represents optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted heterocyclyl from the group consisting of furyl, thienyl, pyrrolyl, pyrazolyl, oxazolyl, isoxazolyl, thiazolyl, pyridinyl or pyrimidinyl.
R³ especially preferably represents methyl, ethyl, isopropyl, isobutyl, tert-butyl, trifluoromethyl, 1,1,2,2-tetrafluoroethyl, pentafluoroethyl, 1-chlorocyclopropyl, phenyl, bromophenyl, nitrophenyl, methoxyphenyl, methylphenyl, naphthyl, 2-furyl, 3-furyl, 2-thienyl or 3-thienyl.
R³ represents, with emphasis, methyl, ethyl, or isopropyl.
R² and R³ furthermore together preferably represent C₃-C₅-alkanediyl(alkylene).
R² and R³ furthermore together particularly preferably represent propane-1,3-diyl(trimethylene), butane-1,4-diyl(tetramethylene) or pentane-1,5-diyl(pentamethylene).
R² and R³ furthermore together very particularly preferably represent propane-1,3-diyl(trimethylene) or butane-1,4-diyl(tetramethylene).
R⁴ preferably represents hydrogen, represents amino, represents in each case optionally hydroxyl-, cyano-, halogen- or C₁-C₄-alkoxy-substituted C₁-C₁₀-alkyl, (C₁-C₁₀-alkyl)carbonyl, C₁-C₁₀-alkylsulfonyl, C₁-C₁₀-alkylamino, (C₁-C₁-alkyl)carbonylamino, C₁-C₁₀-alkylsulfonylamino, or represents in each case optionally cyano- or halogen-substituted C₂-C₁₀-alkenyl, (C₂-C₁₀-alkenyl)carbonyl or C₂-C₁₀-alkynyl.
R⁴ Particularly preferably represents hydrogen, represents amino, represents in each case optionally hydroxyl-, cyano-, fluorine-, chlorine-, bromine-, methoxy-, ethoxy-, n- or isopropoxy-, n-, iso-, sec- or tert-butoxy-substituted methyl, ethyl, n- or isopropyl, n-, iso-, sec- or tert-butyl, acetyl, propionyl, n- or isobutyroyl, methylsulfonyl, ethylsulfonyl, n- or isopropylsulfonyl, n-, iso-, sec- or tert-butylsulfonyl, methylamino, ethylamino, n- or isopropylamino, n-, iso-, sec- or tert-butylamino, acetylamino, propionylamino, n- or isobutyroylamino, methylsulfonylamino, ethylsulfonylamino, n- or isopropylsulfonylamino, n-, iso-, sec- or tert-butylsulfonylamino, or represents in each case optionally cyano-, fluorine-, chlorine- or bromine-substituted propenyl, butenyl, pentenyl, propenylcarbonyl, butenylcarbonyl, pentenylcarbonyl, propynyl, butynyl or pentynyl.
R⁴ very particularly preferably represents hydrogen, represents amino, represents in each case optionally cyano-, fluorine-, chlorine-, bromine-, methoxy-, ethoxy-, n- or isopropoxy-substituted methyl, ethyl, n- or isopropyl, n-, iso- or sec-butyl, acetyl, propionyl, n- or isobutyroyl, methylsulfonyl, ethylsulfonyl, n- or isopropylsulfonyl, methylamino, ethylamino, n- or isopropylamino, acetylamino, propionylamino, methylsulfonylamino, ethylsulfonylamino, n- or isopropylsulfonylamino, or represents in each case optionally fluorine-, chlorine- or bromine-substituted propenyl, butenyl, propenylcarbonyl, butenyl-carbonyl, propynyl or butynyl.
R⁴ especially preferably represents hydrogen or methyl.
R⁵ preferably represents hydrogen, represents optionally hydroxyl-, cyano-, halogen- or C₁-C₄-alkoxy-substituted C₁-C₁₀-alkyl, represents in each case optionally cyano- or halogen-substituted C₂-C₁₀-alkenyl or C₂-C₁-alkynyl, represents optionally cyano-, halogen- or C₁-C₄-alkyl-substituted C₃-C₆-cycloalkyl or C₃-C₆-Cycloalkyl-C₁-C₄-alkyl, represents in each case optionally nitro-, cyano-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, phenyl-, phenoxy- or piperazinyl- (which is optionally substituted by C₁-C₄-alkyl or by optionally halogen-, C₁-C₄-alkyl- or C₁-C₄-alkoxy-substituted phenyl) substituted C₆-C₁₀-aryl or C₆-C₁₀-aryl-C₁-C₄-alkyl, or represents in each case optionally nitro-, cyano-, halogen-, C₁-C₄-alkyl-, C₁-C₄-haloalkyl-, C₁-C₄-alkoxy- or C₁-C₄-haloalkoxy-substituted heterocyclyl or heterocyclyl-C₁-C₄-alkyl having up to 6 carbon atoms and at least one heteroatom from the group consisting of N, O, S in the heterocyclyl group.
R⁵ particularly preferably represents hydrogen, represents in each case optionally hydroxyl-, cyano-, fluorine-, chlorine-, methoxy-, ethoxy-, n- or isopropoxy-substituted methyl, ethyl, n- or isopropyl, n-, iso- or sec-butyl, represents in each case optionally cyano-, fluorine-, chlorine- or bromine-substituted propenyl, butenyl, pentenyl, propynyl, butynyl or pentynyl, represents in each case optionally cyano-, fluorine-, chlorine-, bromine-, methyl-, ethyl-, n- or isopropyl-substituted cyclopropyl, cyclobutyl, cyclopentyl, cyclohexyl, cyclopropylmethyl, cyclobutylmethyl, cyclopentylmethyl or cyclohexylmethyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-, methylthio-, ethylthio-, n- or isopropylthio-, n-, iso-, sec- or tert-butylthio-, difluoromethylthio-, trifluoromethylthio-, phenyl-, phenoxy- or piperazinyl- (which is optionally substituted by methyl, ethyl, n- or isopropyl, n-, iso-, sec- or tert-butyl, or by optionally fluorine-, chlorine-, bromine-, methyl-, ethyl-, n- or isopropyl-, n-, iso-, sec- or tert-butyl-, methoxy-, ethoxy-, n- or isopropoxy-, n-, iso-, sec- or tert-butoxy-substituted phenyl) substituted phenyl, naphthyl, benzyl, phenylethyl or naphthylmethyl, or represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted heterocyclyl or heterocyclylalkyl from the group consisting of furyl, furylmethyl, tetrahydrofuryl, tetrahydrofurylmethyl, thienyl, thienylmethyl, pyrrolyl, pyrrolylmethyl, pyrazolyl, pyrazolylmethyl, oxazolyl, oxazolylmethyl, isoxazolyl, isoxazolylmethyl, thiazolyl, thiazolylmethyl, isothiazolyl, oxadiazolyl, thiadiazolyl, pyridinyl, pyridinylmethyl, pyridinylethyl, pyrazinyl, pyridazinyl, pyrimidinyl, pyrimidinylmethyl.
R⁵ very particularly preferably represents hydrogen, represents in each case optionally cyano-, fluorine-, chlorine-, methoxy-, ethoxy-, n- or isopropoxy-substituted methyl, ethyl, n- or isopropyl, n-, iso- or sec-butyl, represents in each case optionally fluorine-, chlorine- or bromine-substituted propenyl, butenyl, propynyl or butynyl, represents in each case optionally fluorine-, chlorine- or methyl-substituted cyclopropyl, cyclopentyl, cyclohexyl, cyclopropylmethyl, cyclopentylmethyl or cyclohexylmethyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, fluoromethoxy-, difluoromethoxy-, trifluoromethoxy-, chlorodifluoromethoxy-, fluoro-ethoxy-, chloroethoxy-, difluoroethoxy-, dichloroethoxy-, fluorochloroethoxy- or trifluoroethoxy-, methylthio-, ethylthio-, n- or isopropylthio-, difluoromethylthio-, trifluoromethylthio-, phenyl-, phenoxy- or piperazinyl- (which is optionally substituted by methyl, ethyl, n- or isopropyl, or by optionally fluorine-, chlorine-, bromine-, methyl-, ethyl-, n- or isopropyl-, methoxy-, ethoxy-, n- or isopropoxy-substituted phenyl) substituted phenyl, benzyl or phenylethyl, or represents in each case optionally nitro-, cyano-, fluorine-, chlorine-bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted heterocyclyl or heterocyclylalkyl from the group consisting of furyl, furylmethyl, tetrahydrofuryl, tetrahydrofurylmethyl, thienyl, thienylmethyl, pyrrolyl, pyrrolylmethyl, pyrazolyl, pyrazolylmethyl, oxazolyl, oxazolylmethyl, isoxazolyl, isoxazolylmethyl, thiazolyl, thiazolylmethyl, isothiazolyl, oxadiazolyl, thiadiazolyl, pyridinyl, pyridinylmethyl, pyridinylethyl, pyrimidinyl, pyrimidinylmethyl.

Saturated or unsaturated hydrocarbon radicals, such as alkyl or alkenyl, can in each case be straight-chain or branched as far as this is possible, including in combination with heteroatoms, such as, for example, in alkoxy.

Optionally substituted radicals may be mono- or polysubstituted, where in the case of polysubstitutions the substituents can be identical or different.

Halogen-substituted radicals, such as, for example, haloalkyl, are mono- or polyhalogenated. In the case of polyhalogenation, the halogen atoms can be identical or different. Here, halogen represents fluorine, chlorine, bromine and iodine, in particular fluorine, chlorine and bromine.

The general or preferred radical definitions given above apply both to the compounds of the formula (I) and, correspondingly, to the starting materials or intermediates required in each case for the preparation. These radical definitions can be combined with one another as desired, i.e. including combinations between the given preferred ranges.

Preference according to the invention is given to the use according to the invention of compounds of the formula (I) which contain a combination of the meanings given above as being preferred. Particular preference according to the invention is given to the use according to the invention of compounds of the formula (I) which contain a combination of the meanings given above as being particularly preferred.

Very particular preference according to the invention is given to the use according to the invention of compounds of the formula (I) which contain a combination of the meanings given above as being very particularly preferred.

Examples of the compounds of the formula (I) to be used according to the invention, some of which are known, are listed in tables 1 and 2 below.

TABLE 1
Examples of compounds of formula (I)

| | |
|---|---|
| | (I) |
| 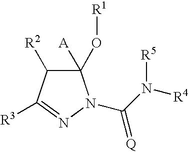 | |

| A | Q | R¹ | R² | R³ | R⁴ | R⁵ |
|---|---|---|---|---|---|---|
| CHF₂ | O | H | H | CH₃ | H | H |
| CF₃ | O | H | H | CH₃ | H | H |
| CF₃ | S | H | H | CH₃ | H | H |
| CF₃ | S | H | H | 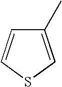 | H | H |
| CF₃ | S | H | H | 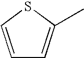 | H | H |
| CF₃ | S | H | H | 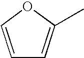 | H | H |
| CF₃ | S | H | H | 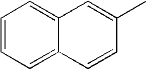 | H | H |
| CF₃ | O | H | H | 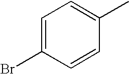 | H | H |
| CF₃ | S | H | H | 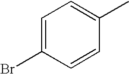 | H | H |
| CF₂CHF₂ | O | H | H | CH₃ | H | H |
| CF₂CHF₂ | O | H | H | C₂H₅ | H | H |
| CF₂CHF₂ | S | H | H | C₂H₅ | H | H |
| CF₂CHF₂ | O | H | H | C₄H₉-t | H | H |
| CF₂CHF₂ | S | H | H | C₄H₉-t | H | H |
| CF₂CHF₂ | O | H | H | CF₂CHF₂ | H | H |
| CF₂CHF₂ | S | H | H | 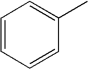 | H | H |
| C₂F₅ | O | H | H | 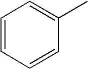 | H | H |
| C₃F₇ | O | H | H | CH₃ | H | H |
| C₃F₇ | S | H | H | CH₃ | H | H |
| C₃F₇ | O | H | H | 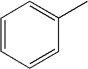 | H | H |
| C₄F₉ | O | H | H | CH₃ | H | H |
| C₈F₁₇ | S | H | H | C₄H₉-n | H | H |
| CF₃ | O | H | H | CH₃ | H | CH₃ |
| CF₃ | O | H | H | C₄H₉-n | H | H |
| CF₃ | O | H | H | 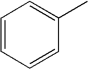 | H | H |
| CF₃ | S | H | H | CH₃ | H | CH₃ |
| CF₃ | S | H | H | C₄H₉-t | H | H |
| CF₃ | S | H | H | 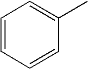 | H | H |
| CF₃ | O | H | H | C₄H₉-i | H | H |
| CF₃ | S | H | H | C₄H₉-i | H | H |
| CCl₃ | O | H | H | 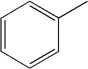 | H | H |
| CCl₃ | O | H | H | CH₃ | H | H |
| CCl₃ | O | H | CH₃ | CH₃ | H | H |
| CCl₃ | O | H | H | 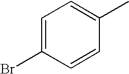 | H | H |
| CCl₃ | O | H | H | 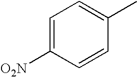 | H | H |
| CCl₃ | O | H | H | 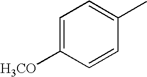 | H | H |
| CCl₃ | S | H | H | CH₃ | H | H |
| CCl₃ | S | H | H | 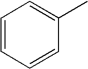 | H | H |
| CCl₃ | S | H | H | 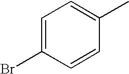 | H | H |
| CCl₃ | S | H | H | 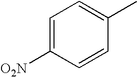 | H | H |
| CCl₃ | S | H | H | 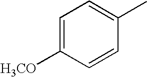 | H | H |
| CF₃ | S | H | H | 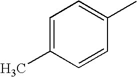 | H | H |
| CF₃ | S | H | H | 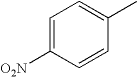 | H | H |

TABLE 2
Further examples of the compounds of the formula (I)

| A | Q | R¹ | R² + R³ | R⁴ | R⁵ |
|---|---|---|---|---|---|
| CHF₂ | S | H | —(CH₂)₄— | H | H |
| CF₃ | S | H | —(CH₂)₄— | H | H |
| CF₂CHF₂ | S | H | —(CH₂)₄— | H | H |
| C₄F₉ | S | H | —(CH₂)₄— | H | H |
| C₆F₁₃ | S | H | —(CH₂)₄— | H | H |
| C₈F₁₇ | S | H | —(CH₂)₄— | H | H |
| CF₃ | O | H | —(CH₂)₄— | H | H |
| C₆F₁₃ | O | H | —(CH₂)₄— | H | H |
| C₈F₁₇ | O | H | —(CH₂)₄— | H | H |
| CF₃ | S | H | —(CH₂)₅— | H | H |
| CF₃ | S | H | —CH₂—C(CH₃)₂—(CH₂)₅— | H | H |

Some of the compounds of the general formula (I) to be used according to the invention are already known (cf. the literature mentioned above in the introduction).

Some of the compounds of the general formula (I) to be used according to the invention are not yet known from the literature.

Thus, the compounds of the general formula (IA)—below—are claimed as novel compounds according to the invention.

A particular group of the compounds to be used according to the invention which, as novel compounds, form part of the subject matter of the present application, is thus represented by the formula (IA) 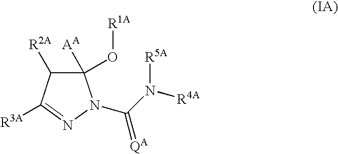
in which
A^{A} represents haloalkyl,
Q^{A} represents oxygen or sulfur,
R^{1A} represents hydrogen or represents in each case optionally substituted alkyl, alkylcarbonyl, cycloalkyl, cycloalkylcarbonyl, aryl, arylcarbonyl, arylalkyl or arylalkylcarbonyl,
R^{2A} represents hydrogen or represents in each case optionally substituted alkyl, alkylcarbonyl, alkoxycarbonyl, cycloalkyl, aryl, arylalkyl or heterocyclyl,
R^{3A} represents in each case optionally substituted alkyl, cycloalkyl, aryl, arylalkyl or heterocyclyl
R^{2A} and R^{3A} furthermore together represent alkanediyl(alkylene),
R^{4A} represents hydrogen, represents amino, or represents in each case optionally substituted alkyl, alkylcarbonyl, alkylsulfonyl, alkylamino, alkylcarbonylamino, alkylsulfonylamino, alkenyl, alkenylcarbonyl or alkynyl, and
R^{5A} represents in each case optionally substituted alkyl having at least 2 carbon atoms, alkenyl, alkynyl, cycloalkyl, cycloalkylalkyl, aryl, arylalkyl, heterocyclyl or heterocyclylalkyl.

Preferred substituents or preferred ranges of the radicals present in the formula (IA) given above are defined below.
A^{A} preferably represents C₁-C₈-haloalkyl.
A^{A} particularly preferably represents fluorine- and/or chlorine-substituted C₁-C₅-alkyl.
A^{A} very particularly preferably represents in each case fluorine- and/or chlorine-substituted methyl, ethyl, n-propyl or isopropyl (such as, for example, trifluoromethyl, difluoromethyl, pentafluoroethyl, heptafluoroisopropyl).
A^{A} especially preferably represents trifluoromethyl.
Q^{A} preferably represents oxygen.
Q^{A} furthermore preferably represents sulfur.
R^{1A} preferably represents hydrogen, represents in each case optionally cyano-, halogen- or C₁-C₄-alkoxy-substituted C₁-C₁₀-alkyl or (C₁-C₁₀-alkyl)carbonyl, represents in each case optionally cyano-, halogen- or C₁-C₄-alkyl-substituted C₃-C₆-Cycloalkyl or (C₃-C₆-cycloalkyl)carbonyl, represents in each case optionally nitro-, cyano-, halogen-, C₁-C₄-alkyl-, C₁-C₄-haloalkyl-, C₁-C₄-alkoxy- or C₁-C₄-haloalkoxy-substituted C₆-C₁₀-aryl or (C₆-C₁₀-aryl)carbonyl, or represents in each case optionally nitro-, cyano-, halogen-, C₁-C₄-alkyl-, C₁-C₄-haloalkyl-, C₁-C₄-alkoxy- or C₁-C₄-haloalkoxy-substituted (C₆-C₁₀-aryl)-C₁-C₄-alkyl or (C₆-C₁₀-aryl-C₁-C₄-alkyl)carbonyl.
R^{1A} particularly preferably represents hydrogen, represents in each case optionally cyano-, fluorine-, chlorine-, bromine-, methoxy-, ethoxy-, n- or isopropoxy-, n-, iso-, sec- or tert-butoxy-substituted methyl, ethyl, n- or isopropyl, n-, iso-, sec- or tert-butyl, n-, iso-, sec-, tert- or neo-pentyl, acetyl, propionyl, n- or isobutyroyl, represents in each case optionally cyano-, fluorine-, chlorine- or bromine-substituted propenyl, butenyl, pentenyl, propenylcarbonyl, butenylcarbonyl or pentenylcarbonyl, represents in each case optionally cyano-, fluorine-, chlorine-, bromine-, methyl-, ethyl-, n- or isopropyl-substituted cyclopropyl, cyclobutyl, cyclopentyl, cyclohexyl, cyclopropylcarbonyl, cyclobutylcarbonyl, cyclopentylcarbonyl or cyclohexylcarbonyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted phenyl, naphthyl, phenylcarbonyl or naphthylcarbonyl, or represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted benzyl, phenylethyl, naphthylmethyl, phenylmethylcarbonyl, phenylethylcarbonyl or naphthylmethylcarbonyl.
R^{1A} very particularly preferably represents hydrogen.
R^{2A} preferably represents hydrogen, represents optionally cyano-, halogen- or C₁-C₄-alkoxy-substituted C₁-C₁₀-alkyl, (C₁-C₁₀-alkyl)carbonyl or (C₁-C₁₀-alkoxy)carbonyl, represents optionally cyano-, halogen- or C₁-C₄-alkyl-substituted C₃-C₆-Cycloalkyl, represents in each case optionally nitro-, cyano-, halogen-, C₁-C₄-alkyl-, C₁-C₄-haloalkyl-, C₁-C₄-alkoxy- or C₁-C₄-haloalkoxy-substituted C₆-C₁₀-aryl or C₆-C₁₀-aryl-C₁-C₄-alkyl, or represents optionally nitro-, cyano-, halogen-, C₁-C₄-alkyl-, C₁-C₄-haloalkyl-, C₁-C₄-alkoxy- or C₁-C₄-haloalkoxy-substituted heterocyclyl having up to 6 carbon atoms and at least one heteroatom from the group consisting of N, O, S.
R^{2A} particularly preferably represents hydrogen, represents in each case optionally cyano-, fluorine-, chlorine-, bromine-, methoxy-, ethoxy-, n- or isopropoxy-, n-, iso-, sec- or tert-butoxy-substituted methyl, ethyl, n- or isopropyl, n-, iso-, sec- or tert-butyl, acetyl, propionyl, n- or isobutyroyl, methoxycarbonyl, ethoxycarbonyl, n- or isopropoxycarbonyl, represents in each case optionally cyano-, fluorine-, chlorine-, bromine-, methyl-, ethyl-, n- or isopropyl-substituted cyclopropyl, cyclobutyl, cyclopentyl or cyclohexyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted phenyl, naphthyl, benzyl or phenylethyl, or represents optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted heterocyclyl from the group consisting of furyl, tetrahydrofuryl, thienyl, pyrrolyl, pyrazolyl, oxazolyl, isoxazolyl, thiazolyl, isothiazolyl, oxadiazolyl, thiadiazolyl, pyridinyl, pyrazinyl, pyridazinyl, pyrimidinyl.
R^{2A} very particularly preferably represents hydrogen, represents in each case optionally cyano-, fluorine-, chlorine-, methoxy-, ethoxy-, n- or isopropoxy-substituted methyl, ethyl, n- or isopropyl, represents methoxycarbonyl or ethoxycarbonyl, represents in each case optionally fluorine-, chlorine- or methyl-substituted cyclopropyl, cyclopentyl or cyclohexyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, fluoromethoxy-, difluoromethoxy-, trifluoromethoxy-, chlorodifluoromethoxy-, fluoro-ethoxy-, chloroethoxy-, difluoroethoxy-, dichloroethoxy-, fluorochloroethoxy- or trifluoroethoxy-substituted phenyl, benzyl or phenylethyl, or represents optionally cyano-, fluorine-, chlorine-, bromine-, methyl-, ethyl-, n- or isopropyl-, fluoromethyl-, chloromethyl-, difluoromethyl-, dichloromethyl-, trifluoromethyl-, trichloromethyl-, methoxy-, ethoxy-, n- or isopropoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted heterocyclyl from the group consisting of furyl, thienyl, pyrrolyl, pyrazolyl, oxazolyl, isoxazolyl, thiazolyl, pyridinyl, pyrimidinyl.
R^{2A} especially preferably represents hydrogen.
R^{3A} preferably represents optionally cyano-, halogen- or C₁-C₄-alkoxy-substituted alkyl having from 1 to 10 carbon atoms, represents optionally cyano-, halogen- or C₁-C₄-alkyl-substituted cycloalkyl having from 3 to 6 carbon atoms, represents in each case optionally nitro-, cyano-, halogen-, C₁-C₄-alkyl-, C₁-C₄-haloalkyl-, C₁-C₄-alkoxy- or, C₁-C₄-haloalkoxy-substituted aryl or arylalkyl having in each case 6 or 10 carbon atoms in the aryl group and optionally from 1 to 4 carbon atoms in the alkyl moiety, or represents optionally nitro-, cyano-, halogen-, C₁-C₄-alkyl-, C₁-C₄-haloalkyl-, C₁-C₄-alkoxy- or C₁-C₄-haloalkoxy-substituted heterocyclyl having up to 6 carbon atoms and at least one heteroatom from the group consisting of N, O, S.
R^{3A} particularly preferably represents in each case optionally cyano-, fluorine-, chlorine-, bromine-, methoxy-, ethoxy-, n- or isopropoxy-, n-, iso-, sec- or tert-butoxy-substituted methyl, ethyl, n- or isopropyl, n-, iso-, sec- or tert-butyl, represents in each case optionally cyano-, fluorine-, chlorine-, bromine-, methyl-, ethyl-, n- or isopropyl-substituted cyclopropyl, cyclobutyl, cyclopentyl or cyclohexyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted phenyl, naphthyl, benzyl or phenylethyl, or represents optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted heterocyclyl from the group consisting of furyl, tetrahydrofuryl, thienyl, pyrrolyl, pyrazolyl, oxazolyl, isoxazolyl, thiazolyl, isothiazolyl, oxadiazolyl, thiadiazolyl, pyridinyl, pyrazinyl, pyridazinyl, pyrimidinyl.
R^{3A} very particularly preferably represents in each case optionally fluorine-, chlorine-, methoxy-, ethoxy-, n- or isopropoxy-substituted methyl, ethyl, n- or isopropyl, isobutyl or tert-butyl, represents in each case optionally fluorine-, chlorine- or methyl-substituted cyclopropyl, cyclopentyl or cyclohexyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted phenyl, naphthyl, benzyl or phenylethyl, or represents optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted heterocyclyl from the group consisting of furyl, thienyl, pyrrolyl, pyrazolyl, oxazolyl, isoxazolyl, thiazolyl, pyridinyl, pyrimidinyl.
R^{3A} especially preferably represents methyl, ethyl, isopropyl, isobutyl, tert-butyl, trifluoromethyl, 1,1,2,2-tetrafluoroethyl, pentafluoroethyl, 1-chlorocyclopropyl, phenyl, bromophenyl, nitrophenyl, methoxyphenyl, methylphenyl, naphthyl, 2-furyl, 3-furyl, 2-thienyl or 3-thienyl.
R^{3A} represents, with emphasis, methyl, ethyl, isopropyl.
R^{2A} and R^{3A} furthermore together preferably represent C₃-C₅-alkanediyl(alkylene).
R^{2A} and R^{3A} furthermore together particularly preferably represent propane-1,3-diyl(trimethylene), butane-1,4-diyl(tetramethylene) or pentane-1,5-diyl(pentamethylene).
R^{2A} and R^{3A} furthermore together very particularly preferably represent propane-1,3-diyl (trimethylene) or butane-1,4-diyl(tetramethylene).
R^{4A} preferably represents hydrogen, represents amino, represents in each case optionally cyano-, halogen- or C₁-C₄-alkoxy-substituted C₁-C₁₀-alkyl, (C₁-C₁₀-alkyl)carbonyl, C₁-C₁₀-alkylsulfonyl, C₁-C₁₀-alkylamino, (C₁-C₁₀-alkyl)carbonylamino or C₁-C₁₀-alkylsulfonylamino, or represents in each case optionally halogen-substituted C₂-C₁₀-alkenyl, (C₂-C₁₀-alkenyl)carbonyl or C₂-C₁₀-alkynyl.
R^{4A} particularly preferably represents hydrogen, represents amino, represents in each case optionally cyano-, fluorine-, chlorine-, bromine-, methoxy-, ethoxy-, n- or isopropoxy-, n-, iso-, sec- or tert-butoxy-substituted methyl, ethyl, n- or isopropyl, n-, iso-, sec- or tert-butyl, acetyl, propionyl, n- or isobutyroyl, methylsulfonyl, ethylsulfonyl, n- or isopropylsulfonyl, n-, iso-, sec- or tert-butylsulfonyl, methylamino, ethylamino, n- or isopropylamino, n-, iso-, sec- or tert-butylamino, acetylamino, propionylamino, n- or isobutyroylamino, methyl-sulfonylamino, ethylsulfonylamino, n- or isopropylsulfonylamino, n-, iso-, sec- or tert-butylsulfonylamino, or represents in each case optionally cyano-, fluorine-, chlorine- or bromine-substituted propenyl, butenyl, pentenyl, propenylcarbonyl, butenylcarbonyl, pentenylcarbonyl, propynyl, butynyl or pentynyl.
R^{4A} very particularly preferably represents hydrogen, represents amino, represents in each case optionally cyano-, fluorine-, chlorine-, bromine-, methoxy-, ethoxy-, n- or isopropoxy-substituted methyl, ethyl, n- or isopropyl, n-, iso- or sec-butyl, acetyl, propionyl, n- or isobutyroyl, methylsulfonyl, ethylsulfonyl, n- or isopropylsulfonyl, methylamino, ethylamino, n- or isopropylamino, acetylamino, propionylamino, methylsulfonylamino, ethylsulfonylamino, n- or isopropylsulfonylamino, or represents in each case optionally fluorine-, chlorine- or bromine-substituted propenyl, butenyl, propenylcarbonyl, butenylcarbonyl, propynyl or butynyl, especially hydrogen.
R^{4A} especially preferably represents hydrogen or methyl.
R^{5A} preferably represents optionally cyano-, halogen- or C₁-C₄-alkoxy-substituted C₂-C₁₀-alkyl, represents in each case optionally halogen-substituted C₂-C₁₀-alkenyl or C₂-C₁₀-alkynyl, represents optionally cyano-, halogen- or C₁-C₄-alkyl-substituted C₃-C₆-cycloalkyl or C₃-C₆-cycloalkyl-C₁-C₄-alkyl, represents in each case optionally nitro-, cyano-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkyl-thio-, phenyl-, phenoxy- or piperazinyl- (which is optionally substituted by C₁-C₄-alkyl or by optionally halogen-, C₁-C₄-alkyl- or C₁-C₄-alkoxy-substituted phenyl) substituted C₆-C₁₀-aryl or C₆-C₁₀-aryl-C₁-C₄-alkyl, or represents in each case optionally nitro-, cyano-, halogen-, C₁-C₄-alkyl-, C₁-C₄-haloalkyl-, C₁-C₄-alkoxy- or C₁-C₄-haloalkoxy-substituted heterocyclyl or heterocyclyl-C₁-C₄-alkyl having up to 6 carbon atoms and at least one heteroatom from the group consisting of N, O, S in the heterocyclyl group.
R^{5A} particularly preferably represents in each case optionally hydroxyl-, cyano-, fluorine-, chlorine-, methoxy-, ethoxy-, n- or isopropoxy-substituted ethyl, n- or isopropyl, n-, iso- or sec-butyl, represents in each case optionally cyano-, fluorine-, chlorine- or bromine-substituted propenyl, butenyl, pentenyl, propynyl, butynyl or pentynyl, represents in each case optionally cyano-, fluorine-, chlorine-, bromine-, methyl-, ethyl-, n- or isopropyl-substituted cyclopropyl, cyclobutyl, cyclopentyl, cyclohexyl, cyclopropylmethyl, cyclo-butylmethyl, cyclopentylmethyl or cyclohexylmethyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-, methylthio-, ethylthio-, n- or isopropylthio-, n-, iso-, sec- or tert-butylthio-, difluoromethylthio-, trifluoromethylthio-, phenyl-, phenoxy- or piperazinyl- (which is optionally substituted by methyl, ethyl, n- or isopropyl, n-, iso-, sec- or tert-butyl, or by optionally fluorine-, chlorine-, bromine-, methyl-, ethyl-, n- or isopropyl-, n-, iso-, sec- or tert-butyl-, methoxy-, ethoxy-, n- or isopropoxy-, n-, iso-, sec- or tert-butoxy-substituted phenyl) substituted phenyl, naphthyl, benzyl, phenylethyl or naphthylmethyl, or represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted heterocyclyl or heterocyclylalkyl from the group consisting of furyl, furylmethyl, tetrahydrofuryl, tetrahydrofurylmethyl, thienyl, thienylmethyl, pyrrolyl, pyrrolylmethyl, pyrazolyl, pyrazolylmethyl, oxazolyl, oxazolylmethyl, isoxazolyl, isoxazolylmethyl, thiazolyl, thiazolylmethyl, isothiazolyl, oxadiazolyl, thiadiazolyl, pyridinyl, pyridinylmethyl, pyridinylethyl, pyrazinyl, pyridazinyl, pyrimidinyl, pyrimidinylmethyl.
R^{5A} very particularly preferably represents in each case optionally cyano-, fluorine-, chlorine-, methoxy-, ethoxy-, n- or isopropoxy-substituted ethyl, n- or isopropyl, n-, iso- or sec-butyl, represents in each case optionally fluorine-, chlorine- or bromine-substituted propenyl, butenyl, propynyl or butynyl, represents in each case optionally fluorine-, chlorine- or methyl-substituted cyclopropyl, cyclopentyl, cyclohexyl, cyclopropylmethyl, cyclopentylmethyl or cyclohexylmethyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-, methylthio-, ethylthio-, n- or isopropylthio-, difluoromethylthio-, trifluoromethylthio-, phenyl-, phenoxy- or piperazinyl- (which is optionally substituted by methyl, ethyl, n- or isopropyl, or by optionally fluorine-, chlorine-, bromine-, methyl-, ethyl-, n- or isopropyl-, methoxy-, ethoxy-, n- or isopropoxy-substituted phenyl) substituted phenyl, benzyl or phenylethyl, or represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted heterocyclyl or heterocyclylalkyl from the group consisting of furyl, furylmethyl, tetrahydrofuryl, tetrahydrofurylmethyl, thienyl, thienylmethyl, pyrrolyl, pyrrolylmethyl, pyrazolyl, pyrazolylmethyl, oxazolyl, oxazolyl-methyl, isoxazolyl, isoxazolylmethyl, thiazolyl, thiazolylmethyl, isothiazolyl, oxadiazolyl, thiadiazolyl, pyridinyl, pyridinylmethyl, pyridinylethyl, pyrimidinyl, pyrimidinylmethyl.

Emphasis is given to compounds of the formula (IB) in which 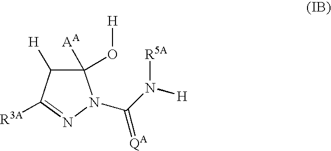
in which
A^{A} represents fluorine- and/or chlorine-substituted C₁-C₈-alkyl,
Q^{A} represents oxygen or sulfur,
R^{3A} represents methyl, ethyl, isopropyl, isobutyl, tert-butyl, 1-chlorocyclopropyl, phenyl, 2-furyl, 3-furyl, 2-thienyl or 3-thienyl,
R^{5A} represents in each case optionally hydroxyl-, cyano-, fluorine-, chlorine-, methoxy-, ethoxy-, n- or isopropoxy-substituted ethyl, n- or isopropyl, n-, iso- or sec-butyl, represents in each case optionally cyano-, fluorine-, chlorine- or bromine-substituted propenyl, butenyl, pentenyl, propynyl, butynyl or pentynyl, represents in each case optionally cyano-, fluorine-, chlorine-, bromine-, methyl-, ethyl-, n- or isopropyl-substituted cyclopropyl, cyclobutyl, cyclopentyl, cyclohexyl, cyclopropylmethyl, cyclobutylmethyl, cyclopentylmethyl or cyclohexylmethyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-, methylthio-, ethylthio-, n- or isopropylthio-, n-, iso-, sec- or tert-butylthio-, difluoromethylthio-, trifluoromethylthio-, phenyl-, phenoxy- or piperazinyl- (which is optionally substituted by methyl, ethyl, n- or isopropyl, n-, iso-, sec- or tert-butyl, or by optionally fluorine-, chlorine-, bromine-, methyl-, ethyl-, n- or isopropyl-, n-, iso-, sec- or tert-butyl-, methoxy-, ethoxy-, n- or isopropoxy-, n-, iso-, sec- or tert-butoxy-substituted phenyl) substituted phenyl, naphthyl, benzyl, phenylethyl or naphthylmethyl, or represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, n-, iso-, sec- or tert-butoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-substituted heterocyclyl or heterocyclylalkyl from the group consisting of furyl, furylmethyl, tetrahydrofuryl, tetrahydrofurylmethyl, thienyl, thienylmethyl, pyrrolyl, pyrrolylmethyl, pyrazolyl, pyrazolylmethyl, oxazolyl, oxazolylmethyl, isoxazolyl, isoxazolylmethyl, thiazolyl, thiazolylmethyl, isothiazolyl, oxadiazolyl, thiadiazolyl, pyridinyl, pyridinylmethyl, pyridinylethyl, pyrazinyl, pyridazinyl, pyrimidinyl, pyrimidinylmethyl.

Particular preference is given to compounds of the formula (IB) in which
A^{A} represents trifluoromethyl, trichloromethyl, difluoromethyl, pentafluoroethyl, heptafluoro-propyl or heptafluoroisopropyl,
Q^{A} represents oxygen or sulfur,
R^{3A} represents methyl, ethyl, isopropyl, isobutyl, tert-butyl, 1-chlorocyclopropyl, phenyl, 2-furyl, 3-furyl, 2-thienyl or 3-thienyl,
R^{5A} represents cyclohexyl, represents in each case optionally nitro-, cyano-, fluorine-, chlorine-, bromine-, iodine-, methyl-, ethyl-, n- or isopropyl-, n-, iso-, sec- or tert-butyl-, fluoromethyl-, chloromethyl-, difluoromethyl-, dichloromethyl-, trifluoromethyl-, trichloromethyl-, methoxy-, ethoxy-, n- or isopropoxy-, fluoromethoxy-, difluoromethoxy-, trifluoromethoxy-, chlorodifluoromethoxy-, fluoroethoxy-, chloroethoxy-, difluoroethoxy-, dichloroethoxy-, fluorochloroethoxy- or trifluoroethoxy-, methylthio-, ethylthio-, n- or isopropylthio-, difluoromethylthio-, trifluoromethylthio-, phenyl-, phenoxy- or piperazinyl- (which is optionally substituted by methyl, ethyl, n- or isopropyl, or by optionally fluorine-, chlorine-, bromine-, methyl-, ethyl-, n- or isopropyl-, methoxy-, ethoxy-, n- or isopropoxy-substituted phenyl) substituted phenyl, benzyl, phenylethyl or pyridinyl.

Saturated or unsaturated hydrocarbon radicals, such as alkyl or alkenyl, can in each case be straight-chain or branched as far as this is possible, including in combination with heteroatoms, such as, for example, in alkoxy.

Optionally substituted radicals can be mono- or polysubstituted, where in the case of polysubstitutions the substituents can be identical or different.

Halogen-substituted radicals, such as, for example, haloalkyl, are mono- or polyhalogenated. In the case of polyhalogenation, the halogen atoms can be identical or different. Here, halogen represents fluorine, chlorine, bromine and iodine, in particular fluorine, chlorine and bromine.

Preference according to the invention is given to novel compounds of the formula (IA) which contain a combination of the meanings listed above as being preferred for the radicals Q^{A}, R^{1A}, R², R^{3A}, R^{4A} and R^{5A}.

Particular preference according to the invention is given to novel compounds of the formula (IA) which contain a combination of the meanings listed above as being particularly preferred for the radicals Q^{A}, R^{1A}, R^{2A}, R^{3A}, R^{4A} and R^{5A}.

Very particular preference according to the invention is given to novel compounds of the formula (IA) which contain a combination of the meanings listed above as being very particularly preferred for the radicals Q^{A}, R^{1A}, R^{2A}, R^{3A}, R^{4A} and R^{5A}.

The novel trifluoromethylpyrazolinols and their derivatives of the formula (IA)—and analogously also the pyrazolinols, some of which are known, and their derivatives of the formula (O)—are obtained when
(a) 1,3-dicarbonyl compounds of the general formula (II) 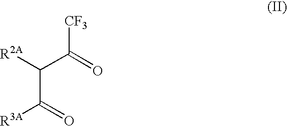
in which R^{2A} and R^{3A} are as defined above,
are reacted with (thio)semicarbazides of the general formula 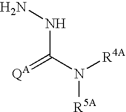
in which Q^{A}, R^{4A} and R^{5A} are as defined above,
if appropriate in the presence of one or more diluents and if appropriate in the presence of
one or more reaction auxiliaries,
   or
   (b) alkoxyalkenyl ketones of the general formula (IV) 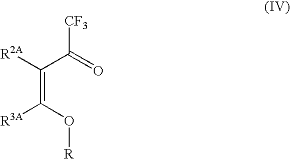
in which
R^{2A} and R^{3A} are as defined above and
R represents alkyl
—and where, in addition to the cis- or Z-configuration illustrated by the formula (IV), the corresponding trans- or E-configuration is also meant to be included—are reacted with (thio)semicarbazides of the general formula (III) 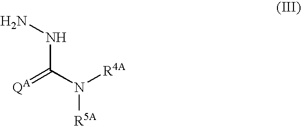
in which Q^{A}, R^{4A} and R^{5A} are as defined above,
if appropriate in the presence of one or more diluents and if appropriate in the presence of one or more reaction auxiliaries,
   or
   (c) pyrazolinols of the general formula (V) 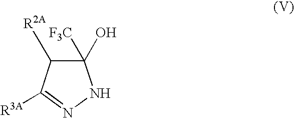
in which R^{2A} and R^{3A} are as defined above
are reacted with aminocarbonyl compounds of the general formula (VI) 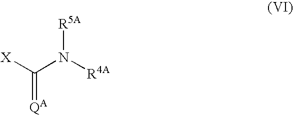
in which
Q^{A}, R^{4A} and R^{5A} areas defined above,
X represents halogen,
or with iso(thio)cyanates of the general formula (VII)
   Q^{A}═C═N—R^{5A} (VII)
in which
Q^{A} is as defined above and
R^{5A} is as defined above, except for H (hydrogen),
if appropriate in the presence of one or more diluents and if appropriate in the presence of one or more reaction auxiliaries,
   and the compounds of the formula (IA) obtained by the processes described under (a), (b) or (c) are, if appropriate, converted by customary methods into other compounds of the formula (I) [or else (IA)].

The compounds of the formula (IA) in which R^{1A} represents H (hydrogen) can be converted by customary methods into corresponding compounds of the formula (IA) in which R^{1A} has the meanings given further above, in general by reacting with compounds of the formula X¹—R^{1A} (VIII) in which R^{1A} is as defined above, except for hydrogen, and X¹ represents halogen, preferably in the presence of reaction auxiliaries, such as, for example, triethylamine or ethyldiisopropylamine, and, if appropriate, in the presence of diluents, such as, for example, tetrahydrofuran, 1,4-dioxane, methyl isobutyl ketone, N,N-dimethylformamide or N,N-dimethylacetamide, at temperatures between 0° C. and 200° C. It is also possible to use diazomethane or trimethyloxonium tetrafluoroborate for the methylation.

Using, for example, 1,1,1-trifluorohexane-2,4-dione and 4-phenylthiosemicarbazide as starting materials, the course of the reaction in the process (a) according to the invention can be illustrated by the formula scheme below:

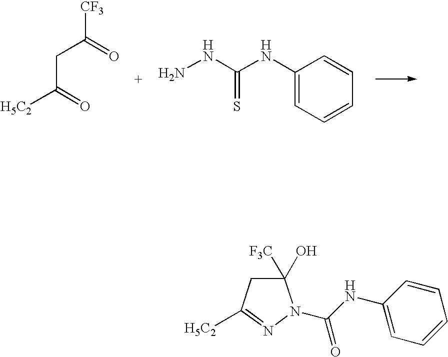

Using, for example, 2-methoxy-5,5,5-trifluoro-2-penten-4-one and 4-ethylsemicarbazide as starting materials, the course of the reaction in the process (b) according to the invention can be illustrated by the formula scheme below:

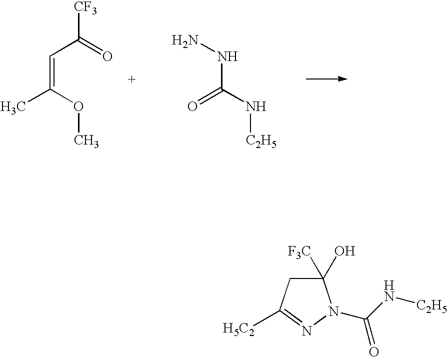

Using, for example, 3-methyl-5-trifluoromethyl-4,5-dihydro-1H-pyrazol-5-ole and cyclohexyl isocyanate as starting materials, the course of the reaction in the process (c) according to the invention can be illustrated by the formula scheme below:

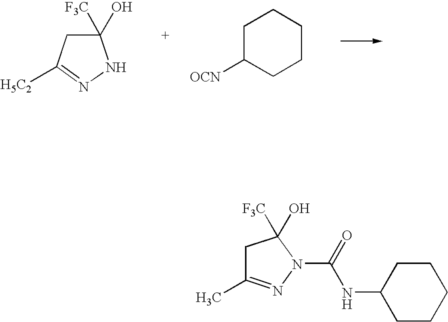

The formula (II) provides a general definition of the 1,3-dicarbonyl compounds to be used as starting materials in the process (a) according to the invention for preparing the compounds of the general formula (IA). In the general formula ([1], R^{2A} and R^{3A} preferably, particularly preferably, very particularly preferably, etc., have those meanings which have already been mentioned above, in connection with the description of the compounds of the general formula (IA) according to the invention, as being preferred, particularly preferred, very particularly preferred, etc., for these radicals.

The starting materials of the general formula (II) are known and/or can be prepared by processes known per se [cf. J. Fluorine Chem. 118 (2002), 135-148; J. Med. Chem. 40 (1997), 1347-1365; Synthesis 11 (1997), 1321-1324; Tetrahedron Lett. 43 (2002), 8701-8705].

The formula (IV) provides a general definition of the alkoxyalkenyl ketones to be used as starting materials in the process (b) according to the invention for preparing the compounds of the general formula (IA). In the general formula (IV), R^{2A} and R^{3A} preferably, particularly preferably, very particularly preferably, etc., have those meanings which have already been mentioned above, in connection with the description of the compounds of the general formula (IA) according to the invention, as being preferred, particularly preferred, very particularly preferred, etc., for these radicals.

The starting materials of the general formula (IV) are known and/or can be prepared by processes known per se [cf. Chem. Ber. 115 (1982), 2766; ibid. 117 (1984), 3270; Chem. Lett. 1976, 499-502; J. Fluorine Chem. 99 (1999), 177-182; Synthesis 1986, 1013-1014; ibid. 1991, 483; Preparation Examples].

The formula (III) provides a general definition of the (thio)semicarbazides further to be used as starting materials in the processes (a) and (b) according to the invention for preparing the compounds of the general formula (IA). In the general formula (III), Q^{A}, R^{4A} and R^{5A} preferably, particularly preferably, very particularly preferably, etc., have those meanings which have already been mentioned above, in connection with the description of the compounds of the general formula (IA) according to the invention, as being preferred, particularly preferred, very particularly preferred, etc., for these radicals.

The starting materials of the general formula (III) are known and/or can be prepared by processes known per se [cf. Arch. Pharm. 335 (2002), 129-134; Bioorg. Med. Chem. Lett. 11 (2001), 1149-1152; Egypt. J. Chem. 41 (1998), 257-266; Org. Synth. Collect. Vol. 5 (1973), 168-170; Pharmazie 55 (2000), 500-502; ibid. 56 (2001), 121-124; Preparation Examples].

The formula (V) provides a general definition of the pyrazolinols to be used as starting materials in the process (c) according to the invention for preparing the compounds of the general formula (IA). In the general formula (V), R^{2A} and R^{3A} preferably, particularly preferably, very particularly preferably, etc., have those meanings which have already been mentioned above, in connection with the description of the compounds of the general formula (IA) according to the invention, as being preferred, particularly preferred, very particularly preferred, etc., for these radicals.

The starting materials of the general formula (V) are known and/or can be prepared by processes known per se [cf. Chem. Het. Comp. 38 (2002), 668-676; J. Fluorine Chem. 94 (1999), 199-203; Russ. Chem. Bull. 51 (2002), 1280-1291; Preparation Examples].

The formula (VI) provides a general definition of the aminocarbonyl compounds further to be used as starting materials in the process (c) according to the invention for preparing the compounds of the general formula (IA). In the general formula (VI), Q^{A}, R^{4A} and R^{5A} preferably, particularly preferably, very particularly preferably, etc., have those meanings which have already been mentioned above, in connection with the description of the compounds of the general formula (IA) according to the invention, as being preferred, particularly preferred, very particularly preferred, etc., for these radicals. X preferably represents chlorine.

Aminocarbonyl compounds of the formula (VI) are known organic chemicals for synthesis.

The formula (VII) provides a general definition of the iso(thio)cyanates alternatively to be used as starting materials in the process (c) according to the invention for preparing the compounds of the general formula (IA). In the general formula (VII), Q^{A} and R^{5A} preferably, particularly preferably, very particularly preferably, etc., have those meanings which have already been mentioned above, in connection with the description of the compounds of the general formula (IA) according to the invention, as being preferred, particularly preferred, very particularly preferred, etc., for these radicals.

Iso(thio)cyanates of the formula (VII) are known organic chemicals for synthesis.

The processes (a), (b) and (c) according to the invention for preparing the compounds of the general formula (IA) are preferably carried out using one or more diluents. Suitable diluents for carrying out the processes (a), (b) and (c) according to the invention are especially inert organic solvents. These include, in particular, aliphatic, alicyclic or aromatic, optionally halogenated hydrocarbons, such as, for example, benzine, benzene, toluene, xylene, chlorobenzene, dichlorobenzene, petroleum ether, hexane, cyclohexane, dichloromethane, chloroform, carbon tetrachloride; ethers, such as diethyl ether, diisopropyl ether, dioxane, tetrahydrofuran or ethylene glycol dimethyl ether or ethylene glycol diethyl ether; ketones, such as acetone, butanone or methyl isobutyl ketone; nitriles, such as acetonitrile, propionitrile or butyronitrile; amides, such as N,N-dimethylformamide, N,N-dimethylacetamide, N-methylformanilide, N-methylpyrrolidone or hexamethylphosphoric triamide; esters, such as methyl acetate or ethyl acetate, sulfoxides, such as dimethyl sulfoxide, alcohols, such as methanol, ethanol, n- or isopropanol, ethylene glycol monomethyl ether, ethylene glycol monoethyl ether, diethylene glycol monomethyl ether, diethylene glycol monoethyl ether, mixtures thereof with water or pure water.

The processes (a), (b) and (c) according to the invention for preparing the compounds of the general formula (I) are, if appropriate, carried out using one or more reaction auxiliaries. Suitable reaction auxiliaries for the processes according to the invention are, in general, the customary inorganic or organic bases or acid acceptors. These preferably include alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate, cesium carbonate or calcium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n-propoxide or isopropoxide, n-butoxide, isobutoxide, sec-butoxide or tert-butoxide or potassium methoxide, ethoxide, n-propoxide or isopropoxide, n-butoxide, isobutoxide, sec-butoxide or tert-butoxide; furthermore also basic organic nitrogen compounds, such as, for example, trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, 1,4-diazabicyclo[2.2.2]octane (DABCO), 1,5-diazabicyclo[4.3.0]non-5-ene (DBN) or 1,8-diazabicyclo[5.4.0]undec-1-ene (DBU). When carrying out the processes (a), (b) and (c) according to the invention, the reaction temperatures can be varied within a relatively wide range. In general, the processes are carried out at temperatures between 0° C. and 150° C., preferably between 10° C. and 120° C.

The processes (a), (b) and (c) according to the invention are generally carried out under atmospheric pressure. However, it is also possible to carry out the processes according to the invention under elevated or reduced pressure—in general between 0.1 bar and 10 bar.

For carrying out the processes (a), (b) and (c) according to the invention, the starting materials are generally employed in approximately equimolar amounts. However, it is also possible to use a relatively large excess of one of the components. The reaction is generally carried out in a suitable diluent in the presence of a reaction auxiliary and the reaction mixture is generally stirred at the required temperature for a number of hours. Work-up is carried out by customary methods (cf. the Preparation Examples).

The processes (a), (b) and (c) are equally suitable for the novel compounds of the formula (IA) and the compounds of the formula (I), some of which are known. To prepare compounds of the formula (I), the meanings of the radicals R^{2A}, R^{3A}, R^{4A} and R^{5A} in the formulae (II) to (VI) correspond to the meanings of R², R³, R⁴ and R⁵.

The active compounds are suitable for protecting plants and plant organs, for increasing the harvest yields, for improving the quality of the harvested material and for controlling animal pests, in particular insects, arachnids and nematodes, which are encountered in agriculture, in forests, in gardens and leisure facilities, in the protection of stored products and of materials, and in the hygiene sector, and have good plant tolerance and favorable toxicity to warm-blooded animals and are tolerated well by the environment. They may be preferably employed as plant protection agents. They are active against normally sensitive and resistant species and against all or some stages of development. The abovementioned pests include:

From the order of the Isopoda, for example, Oniscus asellus, Armadillidium vulgare and Porcellio scaber.

From the order of the Diplopoda, for example, Blaniulus guttulatus.

From the order of the Chilopoda, for example, Geophilus carpophagus and Scutigera spp.

From the order of the Symphyla, for example, Scutigerella immaculata.

From the order of the Thysanura, for example, Lepisma saccharina.

From the order of the Collembola, for example, Onychiurus armatus.

From the order of the Orthoptera, for example, Acheta domesticus, Gryllotalpa spp., Locusta migratoria migratorioides, Melanoplus spp. and Schistocerca gregaria.

From the order of the Blattaria, for example, Blatta orientalis, Periplaneta americana, Leucophaea maderae, Blattella germanica.

From the order of the Dermaptera, for example, Forficula auricularia.

From the order of the Isoptera, for example, Reticulitermes spp.

From the order of the Phthiraptera, for example, Pediculus humanus corporis, Haematopinus spp., Linognathus spp., Trichodectes spp. and Damalinia spp.

From the order of the Thysanoptera, for example, Hercinothrips femoralis, Thrips tabaci, Thrips palmi and Frankliniella accidentalis.

From the order of the Heteroptera, for example, Eurygaster spp., Dysdercus intermedius, Piesma quadrata, Cimex lectularius, Rhodnius prolixus and Triatoma spp.

From the order of the Homoptera, for example, Aleurodes brassicae, Bemisia tabaci, Trialeurodes vaporariorum, Aphis gossypii, Brevicoryne brassicae, Cryptomyzus ribis, Aphis fabae, Aphis pomi, Eriosoma lanigerum, Hyalopterus arundinis, Phylloxera vastatrix, Pemphigus spp., Macrosiphum avenae, Myzus spp., Phorodon humuli, Rhopalosiphum padi, Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp. and Psylla spp.

From the order of the Lepidoptera, for example, Pectinophora gossypiella, Bupalus piniarius, Cheimatobia brumata, Lithocolletis blancardella, Hyponomeuta padella, Plutella xylostella, Malacosoma neustria, Euproctis chrysorrhoea, Lymantria spp., Bucculatrix thurberiella, Phyllocnistis citrella, Agrotis spp., Euxoa spp., Feltia spp., Earias insulana, Heliothis spp., Mamestra brassicae, Panolis flammea, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, Cnaphalocerus spp. and Oulema oryzae.

From the order of the Coleoptera, for example, Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynichus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis, Costelytra zealandica and Lissorhoptrus oryzophilus.

From the order of the Hymenoptera, for example, Diprion spp., Hoplocampa spp., Lasius spp., Monomorium pharaonis and Vespa spp.

From the order of the Diptera, for example, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, Hylemyia spp. and Liriomyza spp.

From the order of the Siphonaptera, for example, Xenopsylla cheopis and Ceratophyllus spp.

From the class of the Arachnida, for example, Scorpio maurus, Latrodectus mactans, Acarus siro, Argas spp., Ornithodoros spp., Dernanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp., Hemitarsonemus spp. and Brevipalpus spp.

The phytoparasitic nematodes include, for example, Pratylenchus spp., Radopholus similis, Ditylenchus dipsaci, Tylenchulus semnipenetrans, Heterodera spp., Globodera spp., Meloidogyne spp., Aphelenchoides spp., Longidorus spp., Xiphinema spp., Trichodorus spp., Bursaphelenchus spp.

The compounds of the formula (I) according to the invention have, in particular, excellent activity against aphids (for example Myzus persicae), beetles and the corresponding larvae (for example Phaedon cochleariae), butterfly caterpillars (for example Spodoptera frugiperda), spidermites (for example Tetranychus urticae), fleas (for example Ctenocephalides felis), flies and the corresponding larvae (for example Lucilia cuprina) and also nematodes (for example Meloidogyne incognita).

If appropriate, the compounds according to the invention can, at certain concentrations or application rates, also be used as herbicides or as safeners for herbicides, or else as microbicides, for example as fungicides, antimycotics and bactericides. If appropriate, they can also be employed as intermediates or precursors for the synthesis of other active compounds.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment according to the invention of the plants and plant parts with the active compounds is carried out directly or by allowing the compounds to act on the surroundings, environment or storage space by the customary treatment methods, for example by immersion, spraying, evaporation, fogging, scattering, painting on, injection and, in the case of propagation material, in particular in the case of seeds, also by applying one or more coats.

The active compounds can be converted to the customary formulations, such as solutions, emulsions, wettable powders, suspensions, powders, dusts, pastes, soluble powders, granules, suspension-emulsion concentrates, natural and synthetic materials impregnated with active compound and microencapsulations in polymeric substances.

These formulations are produced in a known manner, for example by mixing the active compounds with extenders, that is liquid solvents, and/or solid carriers, optionally with the use of surfactants, that is emulsifiers and/or dispersants, and/or foam-formers.

If the extender used is water, it is also possible to employ for example organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulfoxide, and also water.

As solid carriers there are suitable: for example ammonium salts and ground natural minerals such as kaolins, clays, talc, chalk quartz, attapulgite, montmorillonite or diatomaceous earth, and ground synthetic minerals, such as highly disperse silica, alumina and silicates; as solid carriers for granules there are suitable: for example crushed and fractionated natural rocks such as calcite, marble, pumice, sepiolite and dolomite, and also synthetic granules of inorganic and organic meals, and granules of organic material such as sawdust, coconut shells, corn cobs and tobacco stalks; as emulsifiers and/or foam-formers there are suitable: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates and also protein hydrolyzates; as dispersants there are suitable: for example lignosulfite waste liquors and methylcellulose.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations. Other additives can be mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95% by weight of active compound, preferably between 0.5 and 90%.

The active compound according to the invention can be used in its commercially available formulations and in the use forms, prepared from these formulations, as a mixture with other active compounds, such as insecticides, attractants, sterilizing agents, bactericides, acaricides, nematicides, fungicides, growth-regulating substances or herbicides. The insecticides include, for example, phosphoric acid esters, carbamates, carboxylates, chlorinated hydrocarbons, phenylureas and substances produced by microorganisms, inter alia.

Particularly favorable mixing components are, for example, the following compounds:

Fungicides:

2-phenylphenol; 8-hydroxyquinoline sulfate; acibenzolar-5-methyl; aldimorph; amidoflumet; ampropylfos; ampropylfos-potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine; calcium polysulfide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulfamide; flutolanil; flutriafol; folpet; fosetyl-Al; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesilate); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulfocarb; methfuroxam; metiram; metominostrobin; metsulfovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolnitrin; quinconazole; quinoxyfen; quintozene; simeconazole; spiroxamine; sulfur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)—N-[2-[4-[[3-(4-chlorophenyl)-2-propynyl]oxy]-3-methoxyphenyl]ethyl]-3-methyl-2-[(methylsulfonyl)amino]butaneamide; 1-(1-naphthalenyl)-1H-pyrrole-2,5-dione; 2,3,5,6-tetrachloro-4-(methylsulfonyl)pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridinedi-carbonitrile; actinovate; cis-1-(4-chlorophenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-(6-methoxy-3-pyridinyl)cyclopropanecarboxamide; N-butyl-8-(1,1-dimethylethyl)-1-oxa-spiro[4,5]decane-3-amine; sodium tetrathiocarbonate; and copper salts and preparations, such as Bordeaux mixture; copper hydroxide; copper naphthenate; copper oxychloride; copper sulfate; cufraneb; copper oxide; mancopper; oxine-copper.

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

Insecticides/Acaricides/Nematicides: abamectin, ABG-9008, acephate, acequinocyl, acetamiprid, acetoprole, acrinathrin, AKD-1022, AKD-3059, AKD-3088, alanycarb, aldicarb, aldoxycarb, allethrin, alpha-cypermethrin (alpha-methrin), amidoflumet, aminocarb, amitraz, avermectin, AZ-60541, azadirachtin, azamethiphos, azin-phos-methyl, azinphos-ethyl, azocyclotin, Bacillus popilliae, Bacillus sphaericus, Bacillus subtilis, Bacillus thuringiensis, Bacillus thuringiensis strain EG-2348, Bacillus thuringiensis strain GC-91, Bacillus thuringiensis strain NCTC-11821, baculoviruses, Beauveria bassiana, Beauveria tenella, bendothiaz, bendiocarb, benfuracarb, bensultap, benzoximate, beta-cyfluthri, beta-cypermethri, bi-fenazate, bifenthrin, binapacryl, bioallethrin, bioallethrin-S-cyclopentyl-isomer, bioethanomethrin, biopermeibrin, bioresmethrin, bistrifluoron, BPMC, brofenprox, bromophos-ethyl, bromopropylate, bromfenvinfos(-methyl), BTG-504, BTG-505, bufencarb, buprofezin, butathiofos, butocarboxim, butoxycarboxim, butylpyridaben, cadusafos, camphechlor, carbaryl, carbofuran, carbophenothion, carbosulfan, cartap, CGA-50439, chinomethionat, chlordane, chlordimeform, chloethocarb, chlorethoxyfos, chlorfenapyr, chlorfenvinphos, chlorfluazuron, chlormephos, chlorobenzilate, chloropicrin, chlorproxyfen, chlorpyrifos-methyl, chlorpyrifos(-ethyl), chlovaporthrin, chromafenozide, cis-cypermethrin, cis-resmethrin, cis-permeithrin, clocythrin, cloethocarb, clofentezine, clothianidin, clothiazoben, codlemone, coumaphos, cyanofenphos, cyanophos, cycloprene, cycloprothrin, Cydia pomonella, cyfluthrin, cyhalothrin, cyhexatin, cypermethrin, cyphenothrin (1R-trans-isomer), cyromazine, DDT, deltamefirin, demeton-S-methyl, demeton-S-methylsulphone, diafenthiuron, dialifos, diazinon, dichlofenthion, dichlorvos, dicofol, dicrotophos, dicyclanil, diflubenzuron, dimefluthrin, dimethoate, dimethylvinphos, dinobuton, dinocap, dinotefuran, diofenolan, disulfoton, docusat-sodium, dofenapyn, DOWCO-439, eflusilanate, emamectin, emamectin-benzoate, empenthrin (1R-isomer), endosulfan, Entomopthora spp., EPN, esfenvalerate, ethiofencarb, ethiprole, ethion, ethoprophos, etofenprox, etoxazole, etrimfos, famphur, fenamiphos, fenazaquin, fenbutatin oxide, fenfluthrin, fenitrothion, fenobucarb, fenothiocarb, fenoxacrim, fenoxycarb, fenpropathrin, fenpyrad, fenpyrithrin, fenpyroximate, fensulfothion, fenthion, fentrifanil, fenvalerate, fipronil, flonicamid, fluacrypyrim, fluazuron, flubenzimine, flubrocythrinate, flucycloxuron, flucythrinate, flufenerim, flufenoxuron, flufenprox, flumethrin, flupyrazofos, flutenzin (flufenzine), fluvalinate, fonofos, formetanate, formothion, fosmethilan, fosthiazate, fubfenprox (fluproxyfen), furathiocarb, gamma-cyhalothn, gamma-HCH, gossyplure, grandlure, granulosis viruses, halfenprox, halofenozide, HCH, HCN-801, heptenophos, hexaflumuron, hexythiazox, hydramethylnone, hydroprene, IKA-2002, imidacloprid, imiprothrin, indoxacarb, iodofenphos, iprobenfos, isazofos, isofenphos, isoprocarb, isoxathion, ivermectin, japonilure, kadethrin, nuclear polyhedrosis viruses, kinoprene, lambda-cyhalothrin, lindane, lufenuron, malathion,

1. Acetylcholinesterase (AChE) Inhibitors

1.1 carbamates (for example alanycarb, aldicarb, aldoxycarb, allyxycarb, aminocarb, azamethiphos, bendiocarb, benfuracarb, bufencarb, butacarb, butocarboxim, butoxycarboxim, carbaryl, carbofuran, carbosulfan, chloethocarb, coumaphos, cyanofenphos, cyanophos, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, triazamate, trimethacarb, XMC, xylylcarb)

1.2 organophosphates (for example acephate, azamethiphos, azinphos(-methyl, -ethyl), bromophos-ethyl, bromfenvinfos(-methyl), butathiofos, cadusafos, carbophenothion, chlorethoxyfos, chlorfenvinphos, chlormephos, chlorpyrifos(-methyl/-ethyl), coumaphos, cyanofenphos, cyanophos, chlorfenvinphos, demeton-S-methyl, demeton-S-methylsulphone, dialifos, diazinon, dichlofenthion, dichlorvos/DDVP, dicrotophos, dimethoate, dimethylvinphos, dioxabenzofos, disulfoton, EPN, ethion, ethoprophos, etrimfos, famphur, fenamiphos, fenitrothion, fensulfothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion(-methyl/-ethyl), phenthoate, phorate, phosalone, phosmet, phosphamidon, phosphocarb, phoxim, pirimiphos(-methyl/-ethyl), profenofos, propaphos, propetamphos, prothiofos, prothoate, pyraclofos, pyridaphenthion, pyridathion, quinalphos, sebufos, sulfotep, sulprofos, tebupirimfos, temephos, terbufos, tetrachlorvinphos, thiometon, triazophos, triclorfon, vamidothion)

2. Sodium Channel Modulators/Blockers of Voltage-Gated Sodium Channels

2.1 pyrethroids (for example acrinathrin, allethrin (d-cis-trans, d-trans), beta-cyfluthrin, bifenthrin, bioallethrin, bioallethrin-S-cyclopentyl-isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, DDT, deltamethrin, empenthrin (1R-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans isomer), prallethrin, profluthrin, protrifenbute, pyresmethrin, resmethrin, RU 15525, silafluofen, tau-fluvalinate, tefluthrin, terallethrin, tetramethrin (1R-isomer), tralomethrin, transfluthrin, ZXI 8901, pyrethrins (pyrethrum))

2.2 oxadiazines (for example indoxacarb)

3. Acetylcholine Receptor Agonists/Antagonists

3.1 chloronicotinyls/neonicotinoids (for example acetamiprid, clothianidin, dinotefuran, imidacloprid, nitenpyram, nithiazine, thiacloprid, thiamethoxam)

3.2 nicotine, bensultap, cartap

4. Acetylcholine Receptor Modulators

4.1 spinosyns (for example spinosad)

5. Antagonists of GABA-Gated Chloride Channels

5.1 cyclodiene organochlorines (for example camphechlor, chlordane, endosulfan, gamma-HCH, HCH, heptachlor, lindane, methoxychlor

5.2 fiproles (for example acetoprole, ethiprole, fipronil, vaniliprole)

6. Chloride Channel Activators

6.1 mectins (for example abamectin, avermectin, emamectin, emamectin-benzoate, ivermectin, milbemectin, milbemycin)

7. Juvenile Hormone Mimetics

(for example diofenolan, epofenonane, fenoxycarb, hydroprene, kinoprene, methoprene, pyriproxifen, triprene)

8. Ecdyson Agonists/Disruptors

8.1 diacylhydrazines (for example chromafenozide, halofenozide, methoxyfenozide, tebufenozide)

9. Chitin Biosynthesis Inhibitors

9.1 benzoylureas (for example bistrifluoron, chlofluazuron, diflubenzuron, fluazuron, flucycloxuron, flufenoxuron, hexaflumuron, lufenuron, novaluron, noviflumuron, penfluoron, teflubenzuron, tri-flumuron)

9.2 buprofezin

9.3 cyromazine

10. Inhibitors of Oxidative Phosphorylation, ATP Disruptors

10.1 diafenthiuron

10.2 organotins (for example azocyclotin, cyhexatin, fenbutatin-oxide)

11. Uncouplers of Oxidative Phosphorylation Acting by Interrupting the H-Proton Gradient

11.1 pyrroles (for example chlorfenapyr)

11.2 dinitrophenols (for example binapacryl, dinobuton, dinocap, DNOC)

12. Page-I Electron Transport Inhibitors

12.1 METIs (for example fenazaquin, fenpyroximate, pyrimidifen, pyridaben, tebufenpyrad, tolfenpyrad)

12.2 hydramethylnone

12.3 dicofol

13. Page-II Electron Transport Inhibitors

13.1 rotenone

14. Page-III Electron Transport Inhibitors

14.1 acequinocyl, fluacrypyrim

15. Microbial Disruptors of the Insect Gut Membrane

Bacillus thuringiensis strains

16. Inhibitors of Fat Synthesis

16.1 tetronic acids (for example spirodiclofen, spiromesifen)

16.2 tetramic acids [for example 3-(2,5-dimethylphenyl)-8-methoxy-2-oxo-1-azaspiro[4,5]dec-3-en-4-yl ethyl carbonate (alias: carbonic acid, 3-2,5-dimethylphenyl)-8-methoxy-2-oxo-1-azaspiro[4,5]dec-3-en-4-yl ethyl ester, CAS Reg. No.: 382608-10-8) and carbonic acid, cis-3-(2,5-dimethylphenyl)-8-methoxy-2-oxo-1-azaspiro[4,5]dec-3-en-4-yl ethyl ester (CAS Reg. No.: 203313-25-1)]

17. Carboxamides

(for example flonicamid)

18. Octopaminergic Agonists

(for example amitraz)

19. Inhibitors of Magnesium-Stimulated ATPase

(for example propargite)

20. Phthalamides

(for example N²-[1,1-dimethyl-2-(methylsulfonyl)ethyl]-3-iodo-N′-[2-methyl-4-[1,2,2,2-tetrafluoro-1-(trifluoromethyl)ethyl]phenyl]-1,2-benzenedicarboxamide (CAS Reg. No.: 272451-65-7))

21. Nereistoxin Analogs

(for example thiocyclam hydrogen oxalate, thiosultap-sodium)

22. Biologicals, Hormones or Pheromones

(for example azadirachtin, Bacillus spec., Beauveria spec., codlemone, Metarrhizium spec., Paecilomyces spec., thuringiensin, Verticillium spec.)

23. Active Compounds with Unknown or Unspecific Mechanisms of Action

23.1 fumigants (for example aluminum phosphide, methyl bromide, sulfuryl fluoride)

23.2 selective antifeedants (for example cryolite, flonicamid, pymetrozine)

23.3 mite growth inhibitors (for example clofentezine, etoxazole, hexythiazox)

23.4 amidoflumet, benclothiaz, benzoximate, bifenazate, bromopropylate, buprofezin, chinomethionat, chlordimeform, chlorobenzilate, chloropicrin, clothiazoben, cycloprene, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyridalyl, sulfluramid, tetradifon, tetrasul, triarathene, verbutin,

furthermore the compound 3-methylphenyl propylcarbamate (Tsumacide Z, the compound 3-(5-chloro-3-pyridinyl)-8-(2,2,2-trifluoroethyl)-8-azabicyclo[3.2.1]octane-3-carbonitrile (CAS Reg. No. 185982-80-3) and the corresponding 3-endo-isomer (CAS Reg. No. 185984-60-5) (cf. WO 96/37494, WO 98/25923), and preparations which comprise insecticidally active plant extracts, nematodes, fungi or viruses.

A mixture with other known active compounds, such as herbicides, or with fertilizers and growth regulators, safeners and/or semiochemicals is also possible.

When used as insecticides in their commercially available formulations and in the use forms prepared with these formulations, the active compounds according to the invention can furthermore be present in the form of a mixture with synergists. Synergists are compounds by which the activity of the active compounds is increased without it being necessary for the synergist added to be active itself.

When used as insecticides in their commercially available formulations and in the use forms prepared with these formulations, the active compounds according to the invention can furthermore be present in the form of a mixture with inhibitors which reduce the degradation of the active compound after application in the habitat of the plant, on the surface of parts of plants or in plant tissues.

The active compound content of the use forms prepared from the commercially available formulations can vary within broad ranges. The active compound concentration of the use forms can be from 0.0000001 to 95% by weight of active compound, preferably between 0.0001 and 1% by weight.

They are applied in a customary manner adapted to suit the use forms.

When used against hygiene pests and pests of stored products, the active compound is distinguished by excellent residual action on wood and clay as well as good stability to alkali on limed substrates.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof, are treated. The term “parts” or “parts of plants” or “plant parts” has been explained above.

Particularly preferably, plants of the plant cultivars which are in each case commercially available or in use are treated according to the invention. Plant cultivars are to be understood as meaning plants having new properties (“traits”) and which have been obtained by conventional breeding, by mutagenesis or by recombinant DNA techniques. They can be cultivars, varieties, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably to be treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defense of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), corn, soybeans, potatoes, cotton, tobacco, oilseed rape and also fruit plants (with the fruits apples, pears, citrus fruits and grapes), and particular emphasis is given to corn, soybeans, potatoes, cotton, tobacco and oilseed rape. Traits that are particularly emphasized are increased defense of the plants against insects, arachnids, nematodes and slugs and snails by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c Cry2Ab, Cry3Bb and CryIF and also combinations thereof) (hereinbelow referred to as “Bt plants”). Traits that are also particularly emphasized are the increased defense of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulfonylureas, glyphosate or phosphinotricin (for example the “PAT” gene). The genes which impart the desired traits in question can also be present in combination with one another in the transgenic plants. Examples of “Bt plants” which may be mentioned are corn varieties, cotton varieties, soybean varieties and potato varieties which are sold under the trade names YIELD GARD® (for example corn, cotton, soybeans), KnockOut® (for example corn), StarLink® (for example corn), Bollgard® (cotton), Nucotn® (cotton) and NewLeaf® (potato). Examples of herbicide-tolerant plants which may be mentioned are corn varieties, cotton varieties and soybean varieties which are sold under the trade names Roundup Ready® (tolerance to glyphosate, for example corn, cotton, soybean), Liberty Link®0 (tolerance to phosphinotricin, for example oilseed rape), IMI® (tolerance to imidazolinones) and STS® (tolerance to sulfonylureas, for example corn). Herbicide-resistant plants (plants bred in a conventional manner for herbicide tolerance) which may be mentioned also include the varieties sold under the name Clearfield® (for example corn). Of course, these statements also apply to plant cultivars which have these genetic traits or genetic traits still to be developed, and which will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The active compounds according to the invention are not only active against plant, hygiene and stored-product pests, but also, in the veterinary medicine sector, against animal parasites (ectoparasites), such as ixodid ticks, argasid ticks, scab mites, trombi-culid mites, flies (stinging and sucking), parasitic fly larvae, lice, hair lice, bird lice and fleas. These parasites include:

From the order of the Anoplurida, for example, Haematopinus spp., Linognathus spp., Pediculus spp., Phtirus spp. and Solenopotes spp.

From the order of the Mallophagida and the sub-orders Amblycerina and Ischnocerina, for example, Trimenopon spp., Menopon spp., Trinoton spp., Bovicola spp., Werneckiella spp., Lepikentron spp., Damalina spp., Trichodectes spp. and Felicola spp.;

From the order of the Diptera and the sub-orders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

From the order of the Siphonapterida, for example, Pulex spp., Ctenocephalides spp., Xenopyslla spp. and Ceratophyllus spp.

From the order of the Heteropterida, for example, Cimex spp., Triatoma spp., Rhodnius spp. and Panstrongylus spp.

From the order of the Blattarida, for example, Blatta orientalis, Periplaneta americana, Blattela germanica and Supella spp.

From the sub-class of the Acari (Acarida) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Stemostoma spp. and Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The active compounds of the formula (I) according to the invention are also suitable for controlling arthropods which attack agricultural livestock, such as, for example, cattle, sheep, goats, horses, pigs, donkeys, camels, buffaloes, rabbits, chickens, turkeys, ducks, geese, honeybees, other domestic animals, such as, for example, dogs, cats, cage birds, aquarium fish, and so-called experimental animals, such as, for example, hamsters, guinea-pigs, rats and mice. By combating these arthropods, it is intended to reduce deaths and decreased performances (in meat, milk wool, hides, eggs, honey and the like), so that more economical and simpler animal keeping is made possible by using the active compounds according to the invention.

In the veterinary sector, the active compounds according to the invention are used in a known manner by enteral administration, for example in the form of tablets, capsules, drinks, drenches, granules, pastes, boli, the feed-through method, suppositories, by parenteral administration, such as, for example, by means of injections (intramuscular, subcutaneous, intravenous, intraperitoneal and the like), implants, by nasal application, by dermal administration, for example in the form of dipping or bathing, spraying, pouring-on and spotting-on, washing, dusting, and with the aid of shaped articles which comprise active compound, such as collars, ear tags, tail marks, limb bands, halters, marking devices and the like.

When administered to livestock, poultry, domestic animals and the like, the active compounds of the formula (I) can be used as formulations (for example powders, emulsions, flowables) which comprise the active compounds in an amount of 1 to 80% by weight, either directly or after dilution by a factor of 100 to 10000, or they may be used in the form of a chemical bath.

Furthermore, it has been found that the compounds according to the invention have a potent insecticidal action against insects which destroy industrial materials.

The following insects may be mentioned by way of example and as being preferred, but without any limitation:

Beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec., Tryptodendron spec., Apate monachus, Bostrychus capucins, Heterobostrychus brunneus, Sinoxylon spec., Dinoderus minutus; dermapterans, such as Sirex juvencus, Urocerus gigas, Urocerus gigas taignus, Urocerus augur; termites, such as Kalotermes flavicollis, Cryptotermes brevis, Heterotermes indicola, Reticulitermes flavipes, Reticulitermes santonensis, Reticuliterines lucifugus, Mastotermes darwiniensis, Zootermopsis nevadensis, Coptotermes formosanus; bristletails, such as Lepisma saccharina.

Industrial materials are to be understood as meaning, in the present context, non-live materials, such as, preferably, synthetic materials, glues, sizes, paper and board, leather, wood and timber products, and paint.

The materials to be very particularly preferably protected against attack by insects are wood and timber products.

Wood and timber products which can be protected by the composition according to the invention or mixtures comprising such a composition are to be understood as meaning, for example: construction timber, wooden beams, railway sleepers, bridge components, jetties, wooden vehicles, boxes, pallets, containers, telephone poles, wood cladding, windows and doors made of wood, plywood, particle board, joiner's articles, or wood products which, quite generally, are used in the construction of houses or in joinery.

The active compounds can be used as such, in the form of concentrates or generally customary formulations, such as powders, granules, solutions, suspensions, emulsions or pastes.

The formulations mentioned can be prepared in a manner known per se, for example by mixing the active compounds with at least one solvent or diluent, emulsifier, dispersant and/or binder or fixative, water repellent, if appropriate desiccants and UV stabilizers and, if appropriate, colorants and pigments and other processing auxiliaries.

The insecticidal compositions or concentrates used for the protection of wood and wooden materials comprise the active compound according to the invention in a concentration of 0.0001 to 95% by weight, in particular 0.001 to 60% by weight.

The amount of the compositions or concentrates employed depends on the species and the occurrence of the insects and on the medium. The optimum rate of application can be determined upon use in each case by a test series. However, in general, it suffices to employ 0.0001 to 20% by weight, preferably 0.001 to 10% by weight, of the active compound, based on the material to be protected.

The solvent and/or diluent used is an organochemical solvent or solvent mixture and/or an oily or oil-type organochemical solvent or solvent mixture of low volatility and/or a polar organochemical solvent or solvent mixture and/or water and, if appropriate, an emulsifier and/or wetting agent.

Organochemical solvents which are preferably employed are oily or oil-type solvents having an evaporation number of above 35 and a flashpoint of above 30° C., preferably above 45° C. Substances which are used as such oily and oil-type solvents which have low volatility and are insoluble in water are suitable mineral oils or their aromatic fractions, or mineral-oil-containing solvent mixtures, preferably white spirit, petroleum and/or alkylbenzene.

Substances which are advantageously used are mineral oils with a boiling range of 170 to 220° C., white spirit with a boiling range of 170 to 220° C., spindle oil with a boiling range of 250 to 350° C., petroleum or aromatics of boiling range 160 to 280° C., essence of turpentine and the like.

In a preferred embodiment, liquid aliphatic hydrocarbons with a boiling range of 180 to 210° C. or high-boiling mixtures of aromatic and aliphatic hydrocarbons with a boiling range of 180 to 220° C. and/or spindle oil and/or monochloronaphthalene, preferably α-monochloronaphthalene, are used.

The organic oily or oil-type solvents of low volatility having an evaporation number of above 35 and a flashpoint of above 30° C., preferably above 45° C., can be partially replaced by organochemical solvents of high or medium volatility, with the proviso that the solvent mixture also has an evaporation number of above 35 and a flashpoint of above 30° C., preferably above 45° C., and that the insecticide/fungicide mixture is soluble or emulsifiable in this solvent mixture.

In a preferred embodiment, part of the organochemical solvent or solvent mixture is replaced or an aliphatic polar organochemical solvent or solvent mixture. Substances which are preferably used are aliphatic organochemical solvents having hydroxyl and/or ester and/or ether groups, such as, for example, glycol ethers, esters and the like.

The organochemical binders used within the scope of the present invention are the synthetic resins and/or binding drying oils which are known per se and can be diluted with water and/or are soluble or dispersible or emulsifiable in the organochemical solvents employed, in particular binders composed of, or comprising, an acrylate resin, a vinyl resin, for example polyvinyl acetate, polyester resin, polycondensation or polyaddition resin, polyurethane resin, alkyd resin or modified alkyd resin, phenol resin, hydrocarbon resin, such as indene/cumarone resin, silicone resin, drying vegetable and/or drying oils and/or physically drying binders based on a natural and/or synthetic resin.

The synthetic resin used as the binder can be employed in the form of an emulsion, dispersion or solution. Up to 10% by weight of bitumen or bituminous substances can also be used as binders. In addition, colorants, pigments, water repellents, odor-masking substances and inhibitors or anticorrosives known per se and the like can also be employed.

The composition or the concentrate preferably comprises, in accordance with the invention, at least one alkyd resin or modified alkyd resin and/or a drying vegetable oil as the organochemical binder. Preferably used according to the invention are alkyd resins with an oil content of over 45% by weight, preferably 50 to 68% by weight.

All or some of the above-mentioned binder can be replaced by a fixative (mixture) or a plasticizer (mixture). These additives are intended to prevent volatilization of the active compounds and crystallization or precipitation. They preferably replace 0.01 to 30% of the binder (based on 100% of binder employed).

The plasticizers are from the chemical classes of the phthalic esters, such as dibutyl phthalate, dioctyl phthalate or benzyl butyl phthalate, the phosphoric esters, such as tributyl phosphate, the adipic esters, such as di-(2-ethylhexyl)adipate, the stearates, such as butyl stearate or amyl stearate, the oleates, such as butyl oleate, the glycerol ethers or relatively high-molecular-weight glycol ethers, glycerol esters and p-toluenesulfonic esters.

Fixatives are chemically based on polyvinyl alkyl ethers, such as, for example, polyvinyl methyl ether, or ketones, such as benzophenone or ethylenebenzophenone.

Particularly suitable as a solvent or diluent is also water, if appropriate as a mixture with one or more of the above-mentioned organochemical solvents or diluents, emulsifiers and dispersants.

Particularly effective protection of wood is achieved by large-scale industrial impregnation processes, for example vacuum, double-vacuum or pressure processes.

If appropriate, the ready-to-use compositions can additionally comprise other insecticides and, if appropriate, additionally one or more fungicides.

Suitable additional components which may be admixed are, preferably, the insecticides and fungicides mentioned in WO 94/29 268. The compounds mentioned in that document are expressly part of the present application.

Very particularly preferred components which may be admixed are insecticides, such as chlorpyriphos, phoxim, silafluofin, alphamethrin, cyfluthrin, cypermethrin, deltamethrin, permethrin, imidacloprid, NI-25, flufenoxuron, hexaflumuron, transfluthrin, thiacloprid, methoxyfenozide, triflumuron, chlothianidin, spinosad, tefluthrin, and fungicides, such as epoxyconazole, hexaconazole, azaconazole, propiconazole, tebuconazole, cyproconazole, metconazole, imazalil, dichlorfluanid, tolylfluanid, 3-iodo-2-propynylbutyl carbamate, N-octyl-isothiazolin-3-one and 4,5-dichloro-N-octylisothiazolin-3-one.

The compounds according to the invention can at the same time be employed for protecting objects which come into contact with salt water or brackish water, in particular hulls, screens, nets, buildings, moorings and signalling systems, against fouling.

Fouling by sessile Oligochaeta, such as Serpulidae, and by shells and species from the Ledamorpha group (goose barnacles), such as various Lepas and Scalpellum species, or by species from the Balanomorpha group (acorn barnacles), such as Balanus or Pollicipes species, increases the frictional drag of ships and, as a consequence, leads to a marked increase in operation costs owing to higher energy consumption and additionally frequent residence in the dry dock.

Apart from fouling by algae, for example Ectocarpus sp. and Ceramium sp., fouling by sessile Entomostraka groups, which come under the generic term Cirripedia (cirriped crustaceans), is of particular importance.

Surprisingly, it has now been found that the compounds according to the invention, alone or in combination with other active compounds, have an outstanding antifouling action.

Using the compounds according to the invention, alone or in combination with other active compounds, allows the use of heavy metals such as, for example, in bis(trialkyltin) sulfides, tri-n-butyltin laurate, tri-n-butyltin chloride, copper(I) oxide, triethyltin chloride, tri-n-butyl-(2-phenyl-4-chlorophenoxy)tin, tributyltin oxide, molybdenum disulfide, antimony oxide, polymeric butyl titanate, phenyl-bispyridine)-bismuth chloride, tri-n-butyltin fluoride, manganese ethylenebisthio-carbamate, zinc dimethyldithiocarbamate, zinc ethylenebisthiocarbamate, zinc salts and copper salts of 2-pyridinethiol 1-oxide, bisdimethyldithiocarbamoylzinc ethylenebisthiocarbamate, zinc oxide, copper(I) ethylenebisdithiocarbamate, copper thiocyanate, copper naphthenate and tributyltin halides to be dispensed with, or the concentration of these compounds to be substantially reduced.

If appropriate, the ready-to-use antifouling paints can additionally comprise other active compounds, preferably algicides, fungicides, herbicides, molluscicides, or other antifouling active compounds.

Preferably suitable components in combination with the antifouling compositions according to the invention are:

algicides such as 2-tert-butylamino-4-cyclopropylamino-6-methylthio-1,3,5-triazine, dichlorophen, diuron, endothal, fentin acetate, isoproturon, methabenzthiazuron, oxyfluorfen, quinoclamine and terbutryn; fungicides such as benzo[b]thiophenecarboxylic acid cyclohexylamide S,S-dioxide, dichlofluanid, fluorfolpet, 3-iodo-2-propynyl butylcarbamate, tolylfluanid and azoles such as azaconazole, cyproconazole, epoxyconazole, hexaconazole, metconazole, propiconazole and tebuconazole; molluscicides such as fentin acetate, metaldehyde, methiocarb, niclosamid, thiodicarb and trimethacarb; Fe chelates; or conventional antifouling active compounds such as 4,5-dichloro-2-octyl-4-isothiazolin-3-one, diiodomethylparatryl sulfone, 2-(N,N-dimethylthiocarbamoylthio)-5-nitrothiazyl, potassium, copper, sodium and zinc salts of 2-pyridinethiol 1-oxide, pyridine-triphenylborane, tetrabutyldistannoxane, 2,3,5,6-tetrachloro-4-(methylsulfonyl)-pyridine, 2,4,5,6-tetrachloroisophthalonitrile, tetramethylthiuram disulfide and 2,4,6-trichlorophenylmaleimide.

The antifouling compositions used comprise the active compound according to the invention of the compounds according to the invention in a concentration of 0.001 to 50% by weight, in particular 0.01 to 20% by weight.

Moreover, the antifouling compositions according to the invention comprise the customary components such as, for example, those described in Ungerer, Chem. Ind. 1985, 37, 730-732 and Williams, Antifouling Marine Coatings, Noyes, Park Ridge, 1973.

Besides the algicidal, fungicidal, molluscicidal active compounds and insecticidal active compounds according to the invention, antifouling paints comprise, in particular, binders.

Examples of recognized binders are polyvinyl chloride in a solvent system, chlorinated rubber in a solvent system, acrylic resins in a solvent system, in particular in an aqueous system, vinyl chloride/vinyl acetate copolymer systems in the form of aqueous dispersions or in the form of organic solvent systems, butadiene/styrene/acrylonitrile rubbers, drying oils such as linseed oil, resin esters or modified hardened resins in combination with tar or bitumens, asphalt and epoxy compounds, small amounts of chlorine rubber, chlorinated polypropylene and vinyl resins.

If appropriate, paints also comprise inorganic pigments, organic pigments or colorants which are preferably insoluble in salt water. Paints may furthermore comprise materials such as rosin to allow controlled release of the active compounds. Furthermore, the paints may comprise plasticizers, modifiers which affect the rheological properties and other conventional constituents. The compounds according to the invention or the above-mentioned mixtures may also be incorporated into self-polishing antifouling systems.

The active compounds are also suitable for controlling animal pests, in particular insects, arachnids and mites, which are found in enclosed spaces such as, for example, dwellings, factory halls, offices, vehicle cabins and the like. They can be employed in domestic insecticide products for controlling these pests alone or in combination with other active compounds and auxiliaries. They are active against sensitive and resistant species and against all development stages. These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalis, Blattella gernanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleoptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga camaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitumi.

From the order of the Anoplura, for example, Pediculus humanus capitis, Pediculus humanus corporis, Phthirus pubis.

From the order of the Heteroptera, for example, Cimex hemipterus, Cimex lectularius, Rhodinus prolixus, Triatoma infestans.

They are used in the household insecticides sector alone or in combination with other suitable active compounds such as phosphoric esters, carbamates, pyrethroids, neonicotinoids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

### PREPARATION EXAMPLES

### Example 1

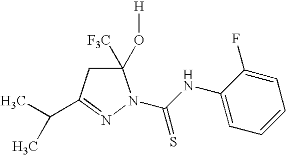 [Process (b)]

A mixture of 0.50 g (2.38 mmol) of 4-ethoxy-1,1,1-trifluoro-5-methyl-hex-3-en-2-one, 0.44 g (2.38 mmol) of 4-(4-fluorophenyl)thiosemicarbazide and 10 ml of methanol is stirred at room temperature (about 20° C.) for 24 hours and then concentrated under reduced pressure. The residue is worked up by column chromatography (silica gel, cyclohexane/ethyl acetate, vol.: 5:1). This gives 0.71 g (85.5% of theory) of N-(2-fluorophenyl)-5-hydroxy-3-(i-propyl)-5-trifluoromethyl-4,5-dihydro-1H-pyrazole-1-carbothioamide as an oil [logP (pH=2.3): 3.86].

### Example 2

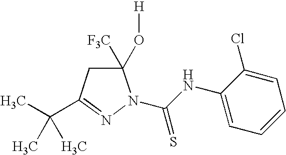 [Process (c)]

A mixture, prepared at room temperature, of 0.50 g (2.0 mmol) of 3-(t-butyl)-5-trifluoromethyl-4,5-dihydro-1H-pyrazol-5-ole, 0.40 g (2.0 mmol) of 2-chlorophenyl isothiocyanate, 6 ml of 1,4-dioxane and 5 drops of triethylamine is, under reflux, heated at the boil for 16 hours and then concentrated under reduced pressure. The residue is then triturated with petroleum ether, and the resulting crystalline product is isolated by filtration with suction. This gives 0.31 g (32% of theory) of N-(2-chlorophenyl)-5-hydroxy-3-t-butyl)-5-trifluoromethyl-4,5-dihydro-1H-pyrazole-1-carbothioamide.

Analogously to Preparation Examples 1 and 2 and in accordance with the general description of the preparation processes according to the invention, it is also possible to prepare, for example, the compounds of the formula (IA) listed in table 3 below.

TABLE 3
Examples of compounds of the formula (IA)

| | |
|---|---|
| | (IA) |
| 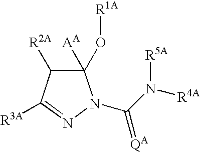 | |

| Ex. | | | | | | | | Physical |
|---|---|---|---|---|---|---|---|---|
| No. | A^{A} | Q^{A} | R^{1A} | R^{2A} | R^{3A} | R^{4A} | R^{5A} | data |
| 3 | CF₃ | O | H | H | C₄H₉-i | H | 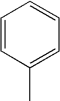 | logP = 3.72^{a)} |
| 4 | CF₃ | O | H | H | C₄H₉-i | H | 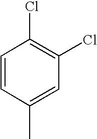 | logP = 4.63^{a)} |
| 5 | CF₃ | O | H | H | C₄H₉-i | H | 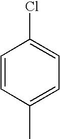 | logP = 4.18^{a)} |
| 6 | CF₃ | O | H | H | C₄H₉-i | H | 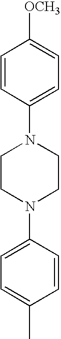 | m.p.: 191° C. |
| 7 | CF₃ | S | H | H | C₄H₉-i | H | 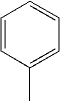 | logP = 4.31^{a)} |
| 8 | CF₃ | S | H | H | C₄H₉-i | H | 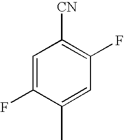 | m.p.: 141° C. |
| 9 | CF₃ | S | H | H | C₄H₉-i | H | 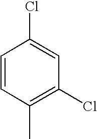 | m.p.: 121° C. |
| 10 | CF₃ | S | H | H | C₄H₉-i | H | 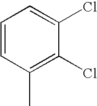 | m.p.: 115° C. |
| 11 | CF₃ | S | H | H | C₄H₉-i | H | 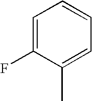 | logP = 4.23^{a)} |
| 12 | CF₃ | O | H | H | C₂H₅ | H | 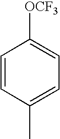 | logP = 3.74^{a)} |
| 13 | CF₃ | O | H | H | C₂H₅ | H | 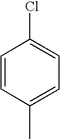 | logP = 3.41^{a)} |
| 14 | CF₃ | S | H | H | C₂H₅ | H | 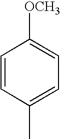 | logP = 3.47^{a)} |
| 15 | CF₃ | S | H | H | C₂H₅ | H | 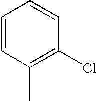 | logP = 3.85^{a)} |
| 16 | CF₃ | S | H | H | C₂H₅ | H | 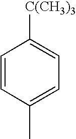 | logP = 4.84^{a)} |
| 17 | CF₃ | O | H | H | C₃H₇-i | H | 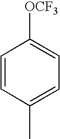 | logP = 4.09^{a)} |
| 18 | CF₃ | O | H | H | C₃H₇-i | H | 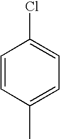 | logP = 3.79^{a)} |
| 19 | CF₃ | S | H | H | C₃H₇-i | H | 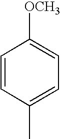 | logP = 3.83^{a)} |
| 20 | CF₃ | S | H | H | C₃H₇-i | H | 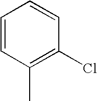 | logP = 4.28^{a)} |
| 21 | CF₃ | S | H | H | C₃H₇-i | H | 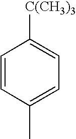 | logP = 5.18^{a)} |
| 22 | CF₃ | O | H | H | C₄H₉-i | H | 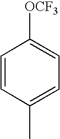 | logP = 4.42^{a)} |
| 23 | CF₃ | S | H | H | C₄H₉-i | H | 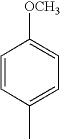 | logP = 4.17^{a)} |
| 24 | CF₃ | S | H | H | C₄H₉-i | H | 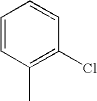 | logP = 4.60^{a)} |
| 25 | CF₃ | S | H | H | C₄H₉-i | H | 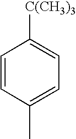 | logP = 5.50^{a)} |
| 26 | CF₃ | O | H | H | 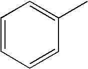 | H | 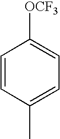 | logP = 4.23^{a)} |
| 27 | CF₃ | S | H | H | C₃H₇-i | H | 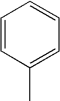 | logP = 3.90^{a)} |
| 28 | CF₃ | O | H | H | C₃H₇-i | H | 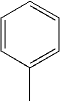 | logP = 3.32^{a)} |
| 29 | CF₃ | S | H | H | C₃H₇-i | H | 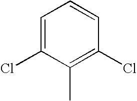 | logP = 4.00^{a)} |
| 30 | CF₃ | S | H | H | C₃H₇-i | H | 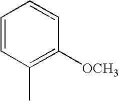 | logP = 4.23^{a)} |
| 31 | CF₃ | S | H | H | C₃H₇-i | H | 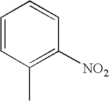 | logP = 4.04^{a)} |
| 32 | CF₃ | S | H | H | C₃H₇-i | H | 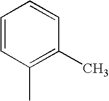 | logP = 4.10^{a)} |
| 33 | CF₃ | S | H | H | C₃H₇-i | H | 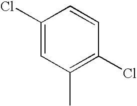 | logP = 4.87^{a)} |
| 34 | CF₃ | S | H | H | C₃H₇-i | H | 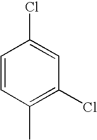 | logP = 4.91^{a)} |
| 35 | CF₃ | S | H | H | C₃H₇-i | H | 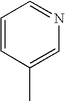 | logP = 1.96^{a)} |
| 36 | CF₃ | S | H | H | C₃H₇-i | H | 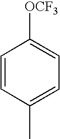 | logP = 4.61^{a)} |
| 37 | CF₃ | O | H | H | C₃H₇-i | H | 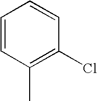 | logP = 4.10^{a)} |
| 38 | CF₃ | S | H | H | C₂H₅ | H | 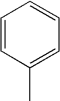 | logP = 3.57^{a)} |
| 39 | CF₃ | O | H | H | C₂H₅ | H | 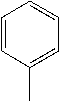 | logP = 2.91^{a)} |
| 40 | CF₃ | S | H | H | C₂H₅ | H | 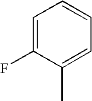 | logP = 3.51^{a)} |
| 41 | CF₃ | S | H | H | C₃H₇-i | H | 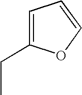 | logP = 3.78^{a)} |
| 42 | CF₃ | S | H | H | C₃H₇-i | H | 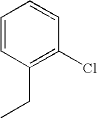 | logP = 4.58^{a)} |
| 43 | CF₃ | S | H | H | C₃H₇-i | H | 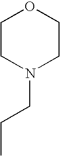 | logP = 1.45^{a)} |
| 44 | CF₃ | S | H | H | C₃H₇-i | H | 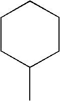 | logP = 4.94^{a)} |
| 45 | CF₃ | S | H | H | C₃H₇-i | H | H | logP = 2.47^{a)} |
| 46 | CF₃ | O | H | H | CH₃ | H | 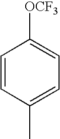 | logP = 3.35^{a)} |
| 47 | CF₃ | O | H | H | C₃H₇-i | H | 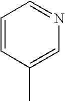 | logP = 1.15^{a)} |
| 48 | CF₃ | O | H | H | C₃H₇-i | H | 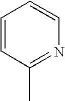 | logP = 1.78^{a)} |
| 49 | CF₃ | O | H | H | C₂H₅ | H | 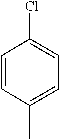 | logP = 3.43^{a)} |
| 50 | CF₃ | O | H | H | C₂H₅ | H | 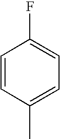 | logP = 2.96^{a)} |
| 51 | CF₃ | O | H | H | C₂H₅ | H | 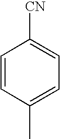 | logP = 2.65^{a)} |
| 52 | CF₃ | O | H | H | 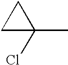 | H | 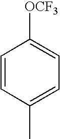 | logP = 4.07^{a)} |
| 53 | CF₃ | S | H | H | C₂H₅ | H | 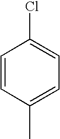 | logP = 4.07^{a)} |
| 54 | CF₃ | S | H | H | C₂H₅ | H | 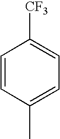 | logP = 4.27^{a)} |
| 55 | CF₃ | S | H | H | C₂H₅ | H | 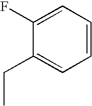 | logP = 3.88^{a)} |
| 56 | CF₃ | S | H | H | C₂H₅ | H | 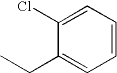 | logP = 4.25^{a)} |
| 57 | CF₃ | S | H | H | C₂H₅ | H | 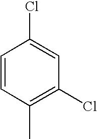 | logP = 4.53^{a)} |
| 58 | CF₃ | O | H | H | C₄H₉-t | H | 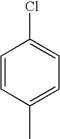 | logP = 4.16^{a)} |
| 59 | CF₃ | O | H | H | C₄H₉-t | H | 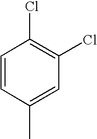 | logP = 4.62^{a)} |
| 60 | CF₃ | O | H | H | C₄H₉-t | H | 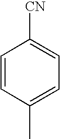 | logP = 3.34^{a)} |
| 61 | CF₃ | S | H | H | C₄H₉-t | H | 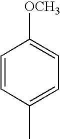 | logP = 3.54^{a)} |
| 62 | CF₃ | S | H | H | C₄H₉-t | H | 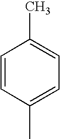 | logP = 4.62^{a)} |
| 63 | CF₃ | S | H | H | C₄H₉-t | H | 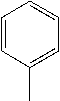 | logP = 4.26^{a)} |
| 64 | CF₃ | S | H | H | C₄H₉-t | H | 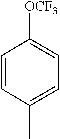 | logP = 4.94^{a)} |
| 65 | CF₃ | O | H | H | C₄H₉-t | H | 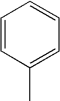 | logP = 4.58^{a)} |
| 66 | CF₃ | O | H | H | C₄H₉-t | H | 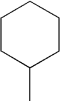 | logP = 3.68^{a)} |
| 67 | CF₃ | O | H | H | C₄H₉-t | H | 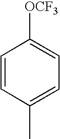 | logP = 4.41^{a)} |
| 68 | CF₃ | O | H | H | C₄H₉-t | H | 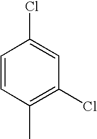 | logP = 5.18^{a)} |
| 69 | CF₃ | O | H | H | C₄H₉-t | H | 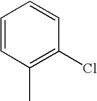 | logP = 4.48^{a)} |
| 70 | CF₃ | O | H | H | C₄H₉-t | H | 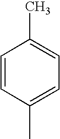 | logP = 4.05^{a)} |
| 71 | CF₃ | O | H | H | C₄H₉-t | H | 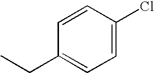 | logP = 3.94^{a)} |
| 72 | CF₃ | O | H | H | C₄H₉-t | H | 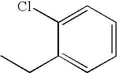 | logP = 3.97^{a)} |
| 73 | CF₃ | S | H | H | C₄H₉-t | H | 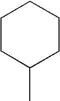 | |
| 74 | CF₃ | S | H | H | C₄H₉-t | H | 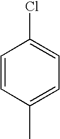 | logP = 4.75^{a)} |
| 75 | CF₃ | S | H | H | C₄H₉-t | H | 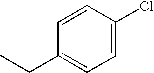 | logP = 4.90^{a)} |
| 76 | CF₃ | O | H | H | C₂H₅ | H | 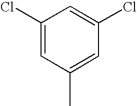 | logP = 4.34^{a)} |
| 77 | CF₃ | O | H | H | C₂H₅ | H | 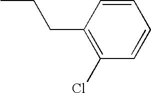 | logP = 3.52^{a)} |
| 78 | CF₃ | S | H | H | 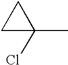 | H | 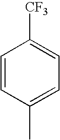 | logP = 4.41^{a)} |
| 79 | CF₃ | S | H | H | 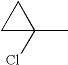 | H | 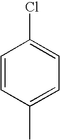 | logP = 4.36^{a)} |
| 80 | CF₃ | O | H | H | 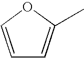 | H | 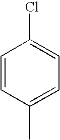 | logP = 3.41^{a)} |
| 81 | CF₃ | S | H | H | 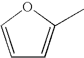 | H | 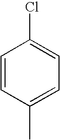 | logP = 3.99^{a)} |
| 82 | CF₃ | O | H | H | 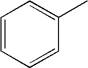 | H | 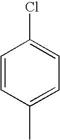 | logP = 3.97^{a)} |
| 83 | CF₃ | S | H | H | 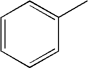 | H | 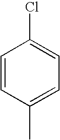 | logP = 4.58^{a)} |
| 84 | CF₃ | S | H | H | C₂H₅ | H | 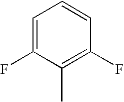 | logP = 3.30^{a)} |
| 85 | CF₃ | S | H | H | C₂H₅ | H | 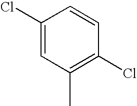 | logP = 4.47^{a)} |
| 86 | CF₃ | S | H | H | C₂H₅ | H | 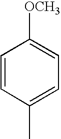 | logP = 3.47^{a)} |
| 87 | CF₃ | S | H | H | C₂H₅ | H | 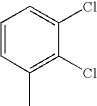 | logP = 4.29^{a)} |
| 88 | CF₃ | S | H | H | C₂H₅ | H | 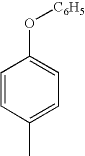 | logP = 4.52^{a)} |
| 89 | CF₃ | S | H | H | C₂H₅ | H | 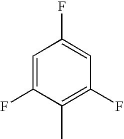 | logP = 3.52^{a)} |
| 90 | CF₃ | S | H | H | C₂H₅ | H | 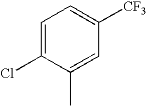 | logP = 4.51^{a)} |
| 91 | CF₃ | S | H | H | C₂H₅ | H | 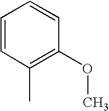 | logP = 3.85^{a)} |
| 92 | CF₃ | S | H | H | C₂H₅ | H | 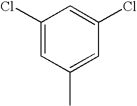 | logP = 4.75^{a)} |
| 93 | CF₃ | S | H | H | C₂H₅ | H | 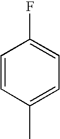 | logP = 3.62^{a)} |
| 94 | CF₃ | S | H | H | C₂H₅ | H | 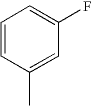 | logP = 3.71^{a)} |
| 95 | CF₃ | S | H | H | C₂H₅ | H | 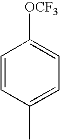 | logP = 4.31^{a)} |
| 96 | CF₃ | S | H | H | C₂H₅ | H | 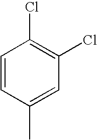 | logP = 4.61^{a)} |
| 97 | CF₃ | O | H | H | C₂H₅ | H | 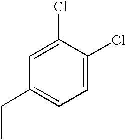 | logP = 4.34^{a)} |
| 98 | CF₃ | S | H | H | C₂H₅ | H | 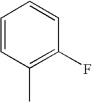 | logP = 3.51^{a)} |
| 99 | CF₃ | S | H | H | C₂H₅ | H | 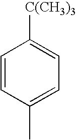 | logP = 4.85^{a)} |
| 100 | CF₃ | S | H | H | CH₃ | H | 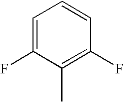 | logP = 2.91^{a)} |
| 101 | CF₃ | S | H | H | CH₃ | H | 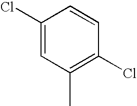 | |
| 102 | CF₃ | S | H | H | CH₃ | H | 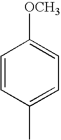 | logP = 3.10^{a)} |
| 103 | CF₃ | S | H | H | CH₃ | H | 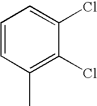 | logP = 3.75^{a)} |
| 104 | CF₃ | S | H | H | CH₃ | H | 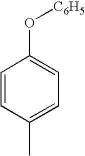 | logP = 4.15^{a)} |
| 105 | CF₃ | S | H | H | CH₃ | H | 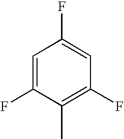 | logP = 3.12^{a)} |
| 106 | CF₃ | O | H | H | CH₃ | H | 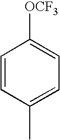 | logP = 3.37^{a)} |
| 107 | CF₃ | S | H | H | CH₃ | H | 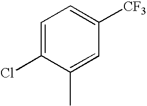 | logP = 3.94^{a)} |
| 108 | CF₃ | S | H | H | CH₃ | H | 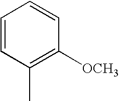 | logP = 3.36^{a)} |
| 109 | CF₃ | S | H | H | CH₃ | H | 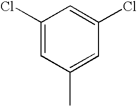 | logP = 4.18^{a)} |
| 110 | CF₃ | S | H | H | CH₃ | H | 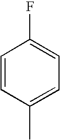 | logP = 2.06^{a)} |
| 111 | CF₃ | S | H | H | CH₃ | H | 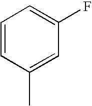 | logP = 3.18^{a)} |
| 112 | CF₃ | S | H | H | CH₃ | H | 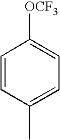 | logP = 3.89^{a)} |
| 113 | CF₃ | S | H | H | CH₃ | H | 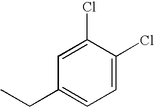 | logP = 4.26^{a)} |
| 114 | CF₃ | O | H | H | CH₃ | H | 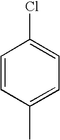 | logP = 3.00^{a)} |
| 115 | CF₃ | O | H | H | CH₃ | H | 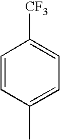 | logP = 3.26^{a)} |
| 116 | CF₃ | O | H | H | CH₃ | H | 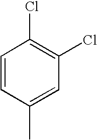 | logP = 3.84^{a)} |
| 117 | CF₃ | O | H | H | CH₃ | H | 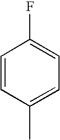 | logP = 2.70^{a)} |
| 118 | CF₃ | S | H | H | CH₃ | H | 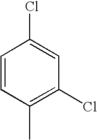 | logP = 3.97^{a)} |
| 119 | CF₃ | S | H | H | CH₃ | H | 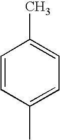 | logP = 3.49^{a)} |
| 120 | CF₃ | S | H | H | CH₃ | H | 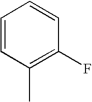 | logP = 3.05^{a)} |
| 121 | CF₃ | S | H | H | CH₃ | H | 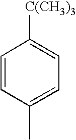 | logP = 4.45^{a)} |
| 122 | CF₃ | S | H | H | CH₃ | H | 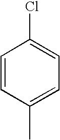 | |
| 123 | CF₃ | S | H | H | CH₃ | H | 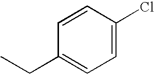 | |
| 124 | CF₃ | S | H | H | CH₃ | H | 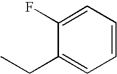 | logP = 3.53^{a)} |
| 125 | CF₃ | S | H | H | CH₃ | H | 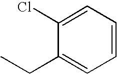 | logP = 3.87^{a)} |
| 126 | CF₃ | S | H | H | CH₃ | H | 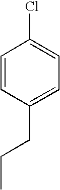 | logP = 4.24^{a)} |
| 127 | CF₃ | S | H | H | CH₃ | H | 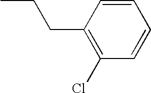 | logP = 3.14^{a)} |
| 128 | CCl₃ | S | H | H | C₄H₉-t | H | 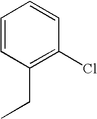 | logP = 5.55^{a)} |
| 129 | CCl₃ | S | H | H | C₄H₉-t | H | 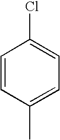 | logP = 4.03^{a)} |
| 130 | CCl₃ | S | H | H | C₄H₉-t | H | 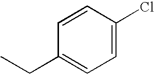 | logP = 2.72^{a)} |
| 131 | CCl₃ | O | H | H | C₄H₉-t | H | 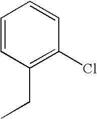 | logP = 4.95^{a)} |
| 132 | C₂F₅ | S | H | H | C₄H₉-t | H | 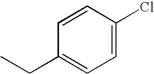 | logP = 5.32^{a)} |
| 133 | C₂F₅ | S | H | H | C₄H₉-t | H | 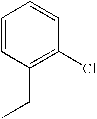 | logP = 5.34^{a)} |
| 134 | C₂F₅ | O | H | H | C₄H₉-t | H | 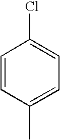 | logP = 4.77^{a)} |
| 135 | C₂F₅ | S | H | H | C₄H₉-t | H | 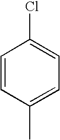 | logP = 5.19^{a)} |
| 136 | C₂F₅ | O | H | H | C₄H₉-t | H | 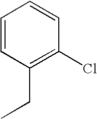 | logP = 4.58^{a)} |
| 137 | C₂F₅ | S | H | H | C₄H₉-t | H | 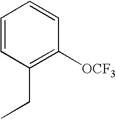 | logP = 5.39^{a)} |
| 138 | C₃F₇ | S | H | H | C₄H₉-t | H | 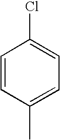 | logP = 5.57^{a)} |
| 139 | C₃H₇ | S | H | H | C₄H₉-t | H | 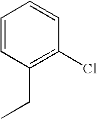 | logP = 5.72^{a)} |
| 140 | C₃F₇ | O | H | H | C₄H₉-t | H | 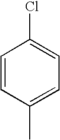 | logP = 5.20^{a)} |
| 141 | C₃F₇ | O | H | H | C₄H₉-t | H | 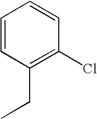 | logP = 5.01^{a)} |
| 142 | CHF₂ | S | H | H | C₄H₉-t | H | 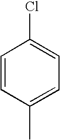 | logP = 4.50^{a)} |
| 143 | CHF₂ | S | H | H | C₄H₉-t | H | 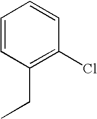 | logP = 4.66^{a)} |
| 144 | CHF₂ | O | H | H | C₄H₉-t | H | 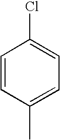 | logP = 3.72^{a)} |
| 145 | CHF₂ | O | H | H | C₄H₉-t | H | 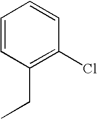 | logP = 3.50^{a)} |
| 146 | CHF₂ | S | H | H | C₄H₉-t | H | 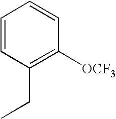 | logP = 4.81^{a)} |

The logP values given in the table were determined in accordance with EEC Directive 79/831 Annex V.A8 by HPLC (High Performance Liquid Chromatography) on a reversed-phase column (C 18). Temperature: 43° C.

(a) Mobile phases for the determination in the acidic range (pH 2.3): 0.1% aqueous phosphoric acid, acetonitrile; linear gradient from 10% acetonitrile to 90% acetonitrile—the corresponding measurement results in table 1 are marked a).

(b) Mobile phases for the determination in the neutral range (pH 7.5): 0.01-molar aqueous phosphate buffer solution, acetonitrile; linear gradient from 10% acetonitrile to 90% acetonitrile—the corresponding measurement results in table 1 are marked b).

Calibration was carried out using unbranched alkan-2-ones (having 3 to 16 carbon atoms) with known logP values (determination of the logP values by the retention times using linear interpolation between two successive alkanones).

The lambdamax values were determined in the maxima of the chromatographic signals using the UV spectra from 200 nm to 400 nm.

Preparation of Starting Materials of the Formula (III)

### Example (III-1)

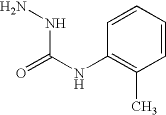 Step 1

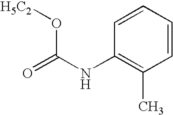

14.5 g (101 mmol) of o-toluidine hydrochloride and 36 ml (207 mmol) of ethyldiisopropylamine are dissolved in 120 ml of dichloromethane and cooled to 0° C., and 10.1 ml (106 mmol) of ethyl chloroformate are added dropwise. The mixture is stirred at room temperature (about 20° C.) for 3 hours, and 100 ml of water are then added. The organic phase is washed with water and saturated sodium chloride solution, dried over sodium sulfate and filtered. From the filtrate, the solvent is carefully distilled off under reduced pressure. This gives 18.2 g (100% of theory) of O-ethyl N-(2-methylphenyl)carbamate as a yellowish oil which is used without further purification for step 2 below.
Step 2

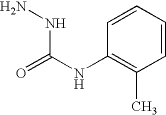

17.2 g (96 mmol) of O-ethyl N-(2-methylphenyl)carbamate and 23.24 ml (480 mmol) of hydrazine monohydrate are stirred at 120° C. for 16 hours. After cooling, the mixture is stored at −14° C. for 16 hours. The resulting crystalline product is isolated by filtration with suction, washed with diethyl ether and pentane and dried under high vacuum. This gives 9.5 g (60% of theory) of 4-(2-methylphenyl)semicarbazide as a colorless solid.

### Example (III-2)

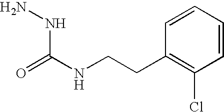

9.8 g (54 mmol) of 2-(2-chlorophenyl)ethyl isocyanate are initially charged in 60 ml of ethanol and cooled to 0° C., and 8.25 ml (59.3 mmol) of 35% strength aqueous hydrazine hydrate solution are slowly added dropwise. Cooling is removed, and the reaction mixture is then stirred for 16 hours (about 20 hours). The resulting solid is isolated by filtration with suction and discarded. From the filtrate, the solvent is carefully distilled off under reduced pressure, and the residue is dried under high vacuum. This gives 10.7 g (93% of theory) of 4-[2-(2-chlorophenyl)ethyl]semicarbazide as a colorless oil which crystallizes to give a white solid.

Preparation of Starting Materials of the Formula (IV)

### Example (IV-1)

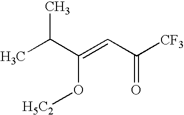 Step 1

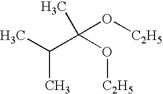

50 g (0.58 mol) of 3-methyl-2-butanone and 86 g (0.58 mol) of triethyl orthoformate are initially charged in 300 ml of ethanol, and a few drops of concentrated hydrochloric acid are added. The mixture is stirred at room temperature (about 20° C.) for 16 hours and then poured into saturated sodium bicarbonate solution and extracted three times with in each case 100 ml of methyl tert-butyl ether, and the organic phase is dried over magnesium sulfate. The solvent is distilled off under reduced pressure and the crude product obtained as residue is used for the next step.

Yield: 68.7 g (64% of theory).
Step 2

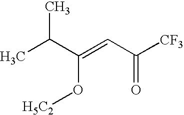

10 g (62 mmol) of the crude product from step 1 are, together with 10 ml of pyridine (124 mmol), dissolved in 60 ml of chloroform. At 0° C., this solution is added dropwise to 17.5 ml (124 mmol) of trifluoroacetic anhydride. After the end of the addition, the mixture is stirred at room temperature (about 20° C.) for a further 17 hours and then washed once with 0.1M hydrochloric acid and subsequently three times with water. The organic phase is dried over magnesium sulfate and filtered. From the filtrate, the solvent is carefully distilled off under reduced pressure. This gives 12 g (92% of theory) of 3-ethoxy-2-methyl-6,6,6-trifluoro-3-hexen-5-one as an amorphous residue.

Preparation of Starting Materials of the Formula (V)

### Example (V-1)

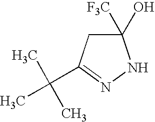

0.31 g (6 mmol) of hydrazine hydrate is initially charged in 15 ml of ethanol, and 1 g (5 mmol) of 1,1,1-trifluoro-5,5-dimethyl-2,4-hexanedione is added dropwise at room temperature (about 20° C.). The mixture is stirred at room temperature for 2 hours and then concentrated under reduced pressure. The residue is triturated with water and extracted repeatedly with ethyl acetate. The organic phase is dried over magnesium sulfate and filtered. From the filtrate, the solvent is carefully distilled off under reduced pressure. This gives: 0.96 g (91.5% of theory) of 3-t-butyl-5-trifluoromethyl-4,5-dihydro-1H-pyrazol-5-ol as an amorphous residue.

### Example (V-2)

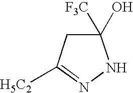

1 g (6 mmol) of 1,1,1-trifluoro-2,4-hexanedione is initially charged in 30 ml of methyl tert-butyl ether, and 0.31 g (6 mmol) of hydrazine hydrate is slowly added at room temperature (about 20° C.). The mixture is stirred for 3.5 hours and then concentrated under reduced pressure. The residue is triturated with water and extracted repeatedly with ethyl acetate. The organic phase is dried over magnesium sulfate and filtered. From the filtrate, the solvent is carefully distilled off under reduced pressure. This gives 0.57 g (52% of theory) of 3-ethyl-5-trifluoromethyl-4,5-dihydro-1H-pyrazol-5-ol as an amorphous residue.

### Use Examples

### Example A

### Meloidogyne Test

| | |
|---|---|
| Solvent: | 7 parts by weight of dimethylformamide |
| Emulsifier: | 2 parts by weight of alkylaryl polyglycol ether |

To produce a suitable preparation of active compound, 1 part by weight of active compound is mixed with the stated amounts of solvent and emulsifier, and the concentrate is diluted with water to the desired concentration.

Containers are filled with sand, solution of active compound, Meloidogyne incognita egg/larvae suspension and lettuce seeds. The lettuce seeds germinate and the plants develop. On the roots, galls are formed.

After the desired period of time, the nematicidal action is determined in % by the formation of galls. 100% means that no galls were found; 0% means that the number of galls on the treated plants corresponds to that of the untreated control.

In this test, for example, the compounds of Preparation Examples 14, 19, 43, 100, 114, 115, 122 and 123 show good activity.

TABLE A
plant-damaging nematodes
Meloidogyne Test

| | | Active | |
|---|---|---|---|
| | | compound | Effect |
| | | concentration | in % |
| Ex. | Active compounds | in ppm | after 14 d |
| (14) | 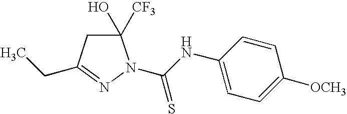 | 20 | 100 |
| (19) | 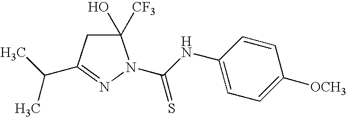 | 20 | 100 |
| (43) | 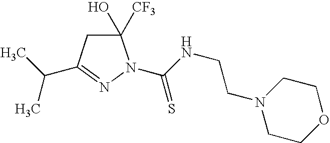 | 20 | 100 |
| (100) | 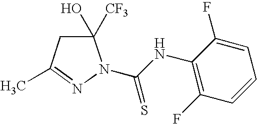 | 20 | 90 |
| (114) | 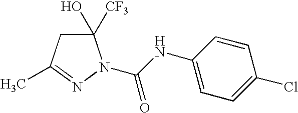 | 20 | 90 |
| (115) | 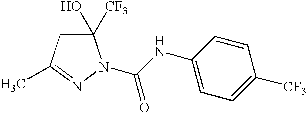 | 20 | 100 |
| (122) | 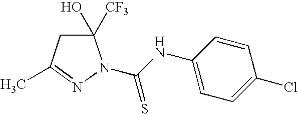 | 20 | 90 |
| (123) | 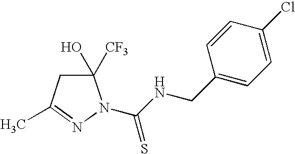 | 20 | 90 |

### Example B

### Myzus Test (Spray Treatment)

| | | |
|---|---|---|
| Solvents: | 78 | parts by weight of acetone |
| | 1.5 | parts by weight of dimethylformamide |
| Emulsifier: | 0.5 | part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Disks of Chinese cabbage (Brassica pekinensis) which are infested by all stages of the green peach aphid (Myzus persicae) are sprayed with a preparation of active compound of the desired concentration.

After the desired period of time, the activity in % is determined. 100% means that all aphids have been killed; 0% means that none of the aphids have been killed.

In this test, for example, the compounds of Preparation Examples 1, 12, 15, 20, 24, 27, 31, 33, 40, 42, 46 and 70 show good activity.

TABLE B
plant-damaging insects
Myzus Test (spray treatment)

| | | Active | Kill |
|---|---|---|---|
| | | compound | rate |
| | | concentration | in % |
| Ex. | Active compounds | in g/ha | after 5 d |
| (1) | 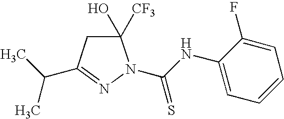 | 500 | 100 |
| (12) | 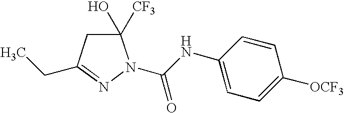 | 500 | 100 |
| (15) | 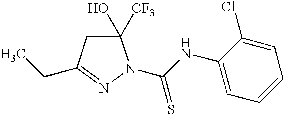 | 500 | 90 |
| (20) | 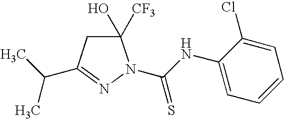 | 500 | 100 |
| (24) | 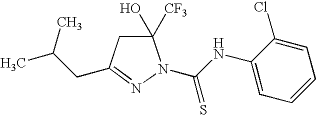 | 500 | 90 |
| (27) | 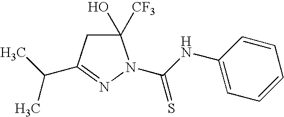 | 500 | 100 |
| (31) | 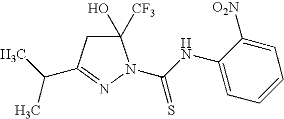 | 500 | 100 |
| (33) | 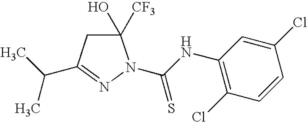 | 500 | 100 |
| (40) | 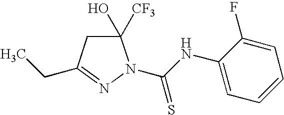 | 500 | 90 |
| (42) | 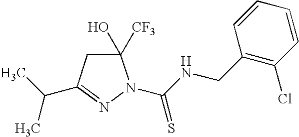 | 500 | 100 |
| (46) | 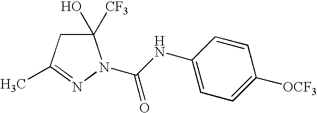 | 500 | 90 |
| (70) | 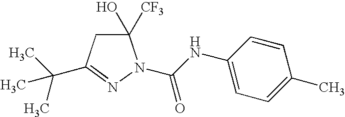 | 100 | 100 |

### Example C

### Phaedon Test (Spray Treatment)

| | | |
|---|---|---|
| Solvents: | 78 | parts by weight of acetone |
| | 1.5 | parts by weight of dimethylformamide |
| Emulsifier: | 0.5 | part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Disks of Chinese cabbage (Brassica pekinensis) are sprayed with a preparation of active compound of the desired concentration and, once they have dried, populated with larvae of the mustard beetle (Phaedon cochleariae).

After the desired period of time, the activity in % is determined. 100% means that all beetle larvae have been killed; 0% means that none of the beetle larvae have been killed.

In this test, for example, the compounds of Preparation Examples 1, 26, 27, 29, 30, 31, 32, 33, 34, 42, 44, 53, 55, 56, 63, 64, 73, 74, 75 show good activity.

TABLE C
plant-damaging insects
Phaedon Test (spray treatment)

| | | Active | Kill |
|---|---|---|---|
| | | compound | rate |
| | | concentration | in % |
| Ex. | Active compounds | in g/ha | after 7 d |
| (1) | 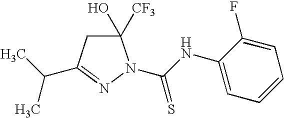 | 500 | 100 |
| (26) | 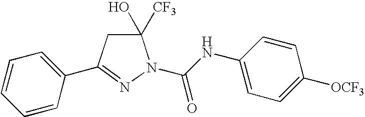 | 500 | 100 |
| (27) | 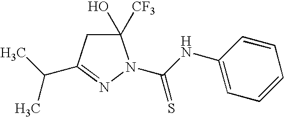 | 500 | 100 |
| (29) | 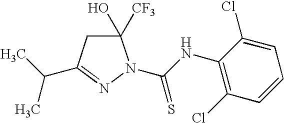 | 500 | 100 |
| (30) | 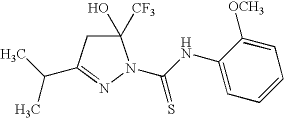 | 500 | 100 |
| (31) | 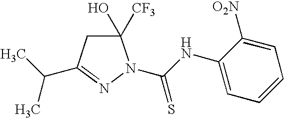 | 500 | 100 |
| (32) | 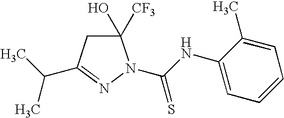 | 500 | 100 |
| (33) | 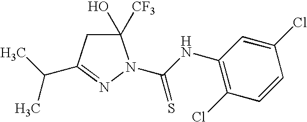 | 500 | 100 |
| (34) | 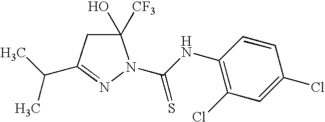 | 500 | 100 |
| (42) | 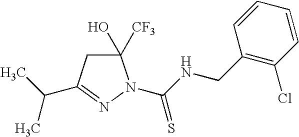 | 500 | 100 |
| (44) | 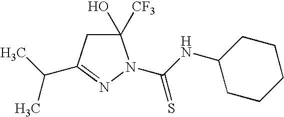 | 500 | 100 |
| (53) | 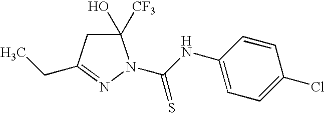 | 500 | 100 |
| (55) | 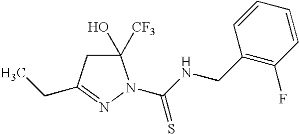 | 500 | 100 |
| (56) | 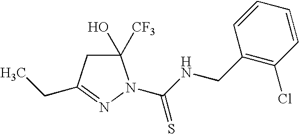 | 500 | 100 |
| (63) | 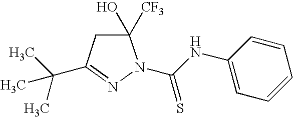 | 100 | 100 |
| (64) | 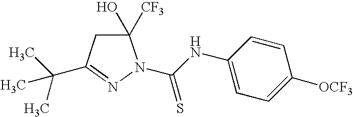 | 100 | 100 |
| (73) | 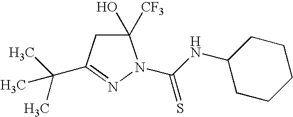 | 100 | 100 |
| (74) | 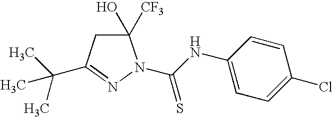 | 100 | 100 |
| (75) | 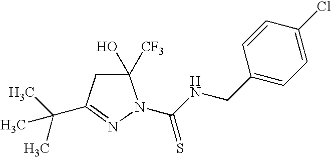 | 100 | 100 |

### Example D

### Spodoptera Frugiperda Test (Spray Treatment)

| | | |
|---|---|---|
| Solvents: | 78 | parts by weight of acetone |
| | 1.5 | parts by weight of dimethylformamide |
| Emulsifier: | 0.5 | part by weight of alkylaryl polyglycol ether |

To produce a suitable preparation of active compound, 1 part by weight of active compound is mixed with, the stated amounts of solvents and emulsifier, and the concentrate is diluted with emulsifier-containing water to the desired concentration.

Disks of corn leaves (Zea mays) are sprayed with a preparation of active compound of the desired concentration and, after they have dried, populated with caterpillars of the armyworm (Spodoptera frugiperda).

After the desired period of time, the activity in % is determined. 100% means that all caterpillars have been killed; 0% means that none of the caterpillars have been killed.

In this test, for example, the compounds of Preparation Examples 1, 20, 24, 26, 27, 29, 31, 32, 42, 44, 55, 63, 64, 73, 74, 75, 83 show good activity.

TABLE D
plant-damaging insects
Spodoptera frugiperda Test (spray treatment)

| | | Active | Kill |
|---|---|---|---|
| | | compound | rate |
| | | concentration | in % |
| Ex. | Active compounds | in g/ha | after 7 d |
| (1) | 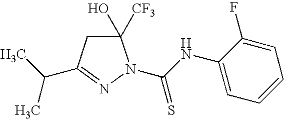 | 500 | 100 |
| (20) | 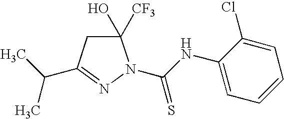 | 500 | 100 |
| (24) | 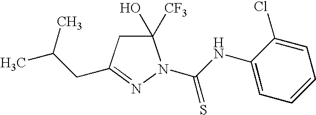 | 500 | 100 |
| (26) | 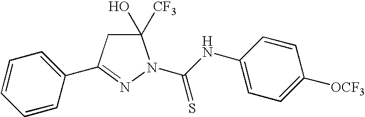 | 500 | 100 |
| (27) | 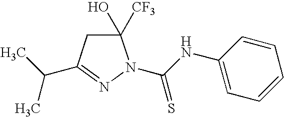 | 500 | 100 |
| (29) | 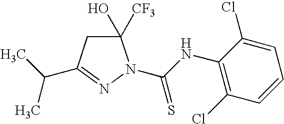 | 500 | 100 |
| (31) | 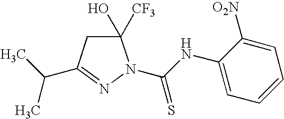 | 500 | 100 |
| (32) | 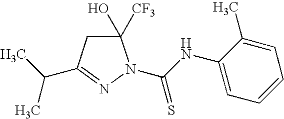 | 500 | 100 |
| (42) | 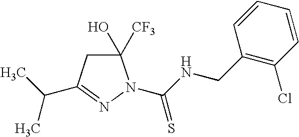 | 500 | 100 |
| (44) | 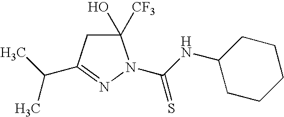 | 500 | 100 |
| (55) | 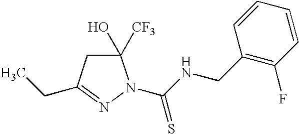 | 500 | 100 |
| (63) | 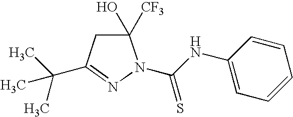 | 100 | 100 |
| (64) | 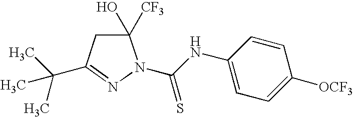 | 100 | 100 |
| (73) | 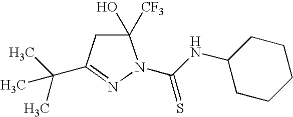 | 100 | 100 |
| (74) | 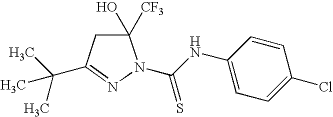 | 100 | 100 |
| (75) | 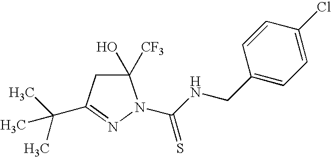 | 100 | 100 |
| (83) | 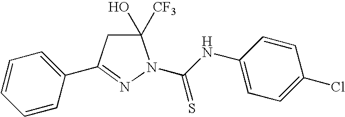 | 500 | 100 |

### Example E

### Tetranychus Test (OP-Resistant/Spray Treatment)

| | | |
|---|---|---|
| Solvents: | 78 | parts by weight of acetone |
| | 1.5 | parts by weight of dimethylformamide |
| Emulsifier: | 0.5 | part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Disks of bean leaves (Phaseolus vulgaris) which are infested by all stages of the greenhouse red spidermite (Tetranychus urticae) are sprayed with a preparation of active compound of the desired concentration.

After the desired period of time, the activity in % is determined. 100% means that all spidermites have been killed; 0% means that none of the spidermites have been killed.

In this test, for example, the compounds of Preparation Examples 1, 22, 24, 27, 30, 34, 42, 44, 55, 56, 74, 75, 106, 118, 126 show good activity.

TABLE E
plant-damaging mites
Tetranychus Test (OP-resistant/spray treatment)

| | | Active | Kill |
|---|---|---|---|
| | | compound | rate |
| | | concentration | in % |
| Ex. | Active compounds | in g/ha | after 5 d |
| (1) | 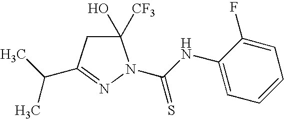 | 100 | 90 |
| (22) | 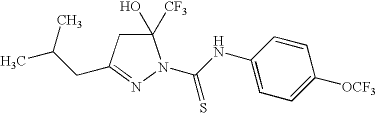 | 500 | 90 |
| (24) | 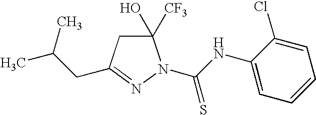 | 500 | 90 |
| (27) | 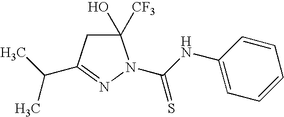 | 100 | 90 |
| (30) | 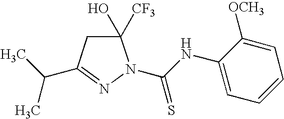 | 500 | 90 |
| (34) | 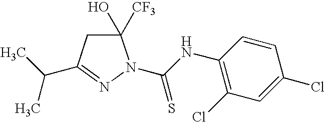 | 500 | 100 |
| (42) | 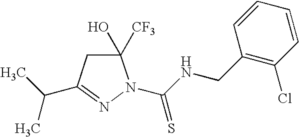 | 500 | 100 |
| (44) | 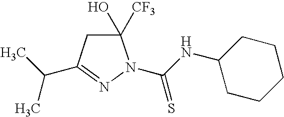 | 500 | 90 |
| (55) | 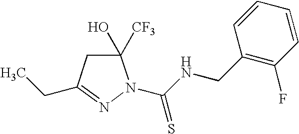 | 100 | 90 |
| (56) | 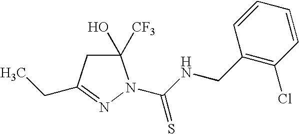 | 100 | 90 |
| (74) | 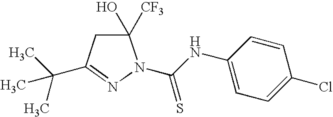 | 100 | 100 |
| (75) | 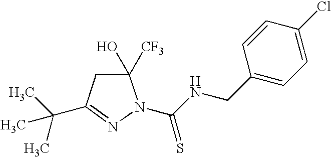 | 100 | 100 |
| (106) | 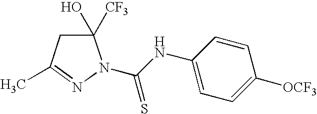 | 100 | 100 |
| (118) | 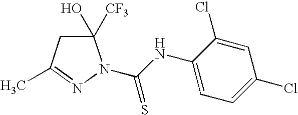 | 100 | 90 |
| (126) | 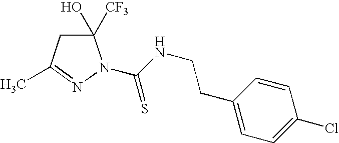 | 100 | 90 |

### Example F

### Test with Cat Fleas/Oral Uptake

Ctenocephalides Felis (Oral)

| | |
|---|---|
| Test animals: | Adults of Ctenocephalides felis |
| Solvent: | Dimethyl sulfoxide (DMSO) |

To produce a suitable formulation, a suitable solution of active compound is prepared from 10 mg of active compound and 0.5 ml of DMSO. 10 μl of this formulation are added to 2 ml of citrated cattle blood and stirred.

20 unfed adult fleas (Ctenocephalides felis, strain “Georgi”) are placed into a chamber (Ø 5 cm) whose top and bottom are closed with gauze. A metal cylinder whose underside is covered with parafilm is placed onto the chamber. The cylinder contains the 2 ml of blood/active compound formulation which can be taken up by the fleas through the parafilm membrane. Whereas the blood is warmed to 37° C., the temperature in the area of the flea chambers is adjusted to room temperature. Controls are mixed with the same volume of DMSO, without addition of a compound.

After 24 h and 48 h, the mortality in % is determined.

Compounds which effect an at least 75% kill of the fleas within 48 h are judged to be effective.

In this test, for example, the compounds of Preparation Examples 29 and 34 show good activity.

TABLE F
Animal parasites
Test with cat fleas/oral uptake

| | | Active | Kill |
|---|---|---|---|
| | | compound | rate |
| | | concentration | in % |
| Ex. | Active compounds | in ppm | after 2 d |
| (29) | 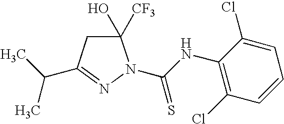 | 100 | 100 |
| (34) | 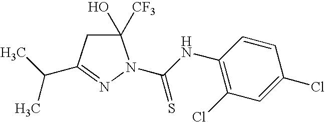 | 100 | 100 |

### Example G

### Fly Larvae Test

Lucilia Cuprina (48 h)

| | |
|---|---|
| Test animals: | Lucilia cuprina larvae |
| Solvent: | Dimethyl sulfoxide |

10 mg of active compound are dissolved in 0.5 ml of dimethyl sulfoxide. To produce a suitable formulation, the solution of active compound is diluted with water to the particular desired concentration.

About 20 Lucilia cuprina larvae are introduced into a test tube which contains about 1 cm³ of horse meat and 0.5 ml of the preparation of active compound to be tested. After 48 hours, the activity of the active compound preparation is determined as % larvae mortality.

In this test, for example, the compound according to Preparation Example 26 shows good activity.

TABLE G
Animal parasites
Fly larvae Test

| | | Active | Kill |
|---|---|---|---|
| | | compound | rate |
| | | concentration | in % |
| Ex. | Active compounds | in ppm | after 2 d |
| (26) | 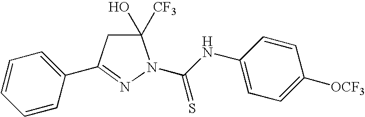 | 100 | 100 |

### Example H

### Diabrotica Balteata Test (Larvae in Soil)

Critical Concentration Test/Soil Insects—Treatment of Transgenic Plants

| | |
|---|---|
| Solvent: | 7 parts by weight of dimethylformamide |
| Emulsifier: | 1 part by weight of alkylaryl polyglycol ether |

To produce a suitable preparation of active compound, 1 part by weight of active compound is mixed with the stated amount of solvent, the stated amount of emulsifier is added and the concentrate is diluted with water to the desired concentration.

The preparation of active compound is poured onto the soil. Here, the concentration of active compound in the preparation is virtually immaterial, only the amount by weight of active compound per volume unit of soil, which is stated in ppm (mg/l), matters. The soil is filled into 0.25 1 pots, and these are allowed to stand at 20° C.

Immediately after the preparation, 5 pregerminated corn grains of the cultivar YIELD GUARD (trademark of Monsanto Comp., USA) are placed into each pot. After 2 days, the appropriate test insects are placed into the treated soil. After a further 7 days, the efficacy of the active compound is determined by counting the corn plants that have emerged (1 plant=20% activity).

### Example J

### Heliothis Virescens Test (Treatment of Transgenic Plants)

| | |
|---|---|
| Solvent: | 7 parts by weight of dimethylformamide |
| Emulsifier: | 1 part by weight of alkylaryl polyglycol ether |

To produce a suitable preparation of active compound, 1 part by weight of active compound is mixed with the stated amount of solvent and the stated amount of emulsifier, and the concentrate is diluted with water to the desired concentration.

Soybean shoots (Glycine max) of the cultivar Roundup Ready (trademark of Monsanto Comp. USA) are treated by being dipped into the preparation of active compound of the desired concentration and are populated with the tobacco budworm Heliothis virescens while the leaves are still moist.

After the desired period of time, the kill of the insects in % is determined. 100% means that all caterpillars have been killed; 0% means that none of the caterpillars have been killed.

1. The use of pyrazolinols and their derivatives of the formula (I) 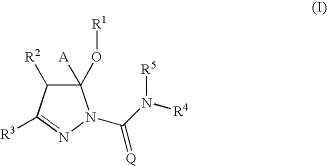
in which
A represents optionally substituted alkyl,
Q represents oxygen or sulfur,
R¹ represents hydrogen or represents in each case optionally substituted alkyl, alkylcarbonyl, alkenyl, alkenylcarbonyl, cycloalkyl, cycloalkylcarbonyl, aryl, aryl-carbonyl, arylalkyl or arylalkylcarbonyl,
R² represents hydrogen or represents in each case optionally substituted alkyl, alkylcarbonyl, alkoxycarbonyl, cycloalkyl, aryl, arylalkyl or heterocyclyl,
R³ represents in each case optionally substituted alkyl, alkenyl, cycloalkyl, aryl, arylalkyl or heterocyclyl,
R² and R³ furthermore together represent alkanediyl(alkylene),
R⁴ represents hydrogen, represents amino, or represents in each case optionally substituted alkyl, alkylcarbonyl, alkylsulfonyl, alkylamino, alkylcarbonylamino, alkylsulfonylamino, alkenyl, alkenylcarbonyl or alkynyl, and
R⁵ represents hydrogen or represents in each case optionally substituted alkyl, alkenyl, alkynyl, cycloalkyl, cycloalkylalkyl, aryl, arylalkyl, heterocyclyl or heterocyclylalkyl, for controlling animal pests.

2. The use of pyrazolinols and their derivatives of the formula (I) as claimed in claim 1, in which
A represents optionally hydroxyl-, cyano-, halogen-, C₁-C₄-alkoxy- or C₁-C₄-halo-alkoxy-substituted C₁-C₈-alkyl,
Q represents oxygen or sulfur,
R¹ represents hydrogen, represents in each case optionally hydroxyl-, cyano-, halogen-, C₁-C₄-alkoxy- or C₁-C₄-haloalkoxy-substituted C₁-C₁₀-alkyl or (C₁-C₁₀-alkyl)-carbonyl, represents in each case optionally cyano- or halogen-substituted C₂-C₁₀-alkenyl or (C₂-C₁₀-alkenyl)carbonyl, represents in each case optionally cyano-, halogen-, C₁-C₄-alkyl- or C₁-C₄-haloalkyl-substituted C₃-C₆-cycloalkyl or (C₃-C₆-cycloalkyl)carbonyl, represents in each case optionally nitro-, cyano-, halogen-, C₁-C₄-alkyl-, C₁-C₄-haloalkyl-, C₁-C₄-alkoxy- or C₁-C₄-haloalkoxy-substituted C₆-C₁₀-aryl or (C₆-C₁₀-aryl)carbonyl, or represents in each case optionally nitro-, cyano-, halogen-, C₁-C₄-alkyl-, C₁-C₄-haloalkyl-, C₁-C₄-alkoxy- or C₁-C₄-haloalkoxy-substituted (C₆-C₁₀-aryl)-C₁-C₄-alkyl or (C₆-C₁₀-aryl-C₁-C₄-alkyl)carbonyl,
R² represents hydrogen, represents optionally cyano-, halogen- or C₁-C₄-alkoxy-substituted C₁-C₁₀-alkyl, (C₁-C₁₀-alkyl)carbonyl or (C₁-C₁₀-alkoxy)carbonyl, represents optionally cyano-, halogen- or C₁-C₄-alkyl-substituted C₃-C₆-cycloalkyl, represents in each case optionally nitro-, cyano-, halogen-, C₁-C₄-alkyl-, C₁-C₄-haloalkyl-, C₁-C₄-alkoxy- or C₁-C₄-haloalkoxy-substituted C₆-C₁₀-aryl or C₆-C₁₀-aryl-C₁-C₄alkyl, or represents optionally nitro-, cyano-, halogen-, C₁-C₄-alkyl-, C₁-C₄-haloalkyl-, C₁-C₄-alkoxy- or C₁-C₄-haloalkoxy-substituted heterocyclyl having up to 6 carbon atoms and at least one heteroatom from the group consisting of N, O, S,
R³ represents optionally cyano-, halogen- or C₁-C₄-alkoxy-substituted C₁-C₁₀-alkyl, represents optionally cyano-, halogen- or C₁-C₄-alkyl-substituted C₃-C₆-cycloalkyl, represents in each case optionally nitro-, cyano-, halogen-, C₁-C₄-alkyl-, C₁-C₄-haloalkyl-, C₁-C₄-alkoxy- or C₁-C₄-haloalkoxy-substituted C₆-C₁₀-aryl or C₆-C₁₀-aryl-C₁-C₄-alkyl, or represents optionally nitro-, cyano-, halogen-, C₁-C₄-alkyl-, C₁-C₄-haloalkyl-, C₁-C₄-alkoxy- or C₁-C₄-haloalkoxy-substituted heterocyclyl having up to 6 carbon atoms and at least one heteroatom from the group consisting of N, O, S,
R² and R³ furthermore together represent C₃-C₅-alkanediyl(alkylene),
R⁴ represents hydrogen, represents amino, represents in each case optionally hydroxyl-, cyano-, halogen- or C₁-C₄-alkoxy-substituted C₁-C₁₀-alkyl, (C₁-C₁₀-alkyl)carbonyl, C₁-C₁₀-alkylsulfonyl, C₁-C₁₀-alkylamino, (C₁-C₁₀-alkyl)carbonylamino, C₁-C₁₀-alkyl-sulfonylamino, or represents in each case optionally cyano- or halogen-substituted C₂-C₁₀-alkenyl, (C₂-C₁₀-alkenyl)carbonyl or C₂-C₁₀-alkynyl,
R⁵ represents hydrogen, represents optionally hydroxyl-, cyano-, halogen- or C₁-C₄-alkoxy-substituted C₁-C₁₀-alkyl, represents in each case optionally cyano- or halogen-substituted C₂-C₁₀-alkenyl or C₂-C₁₀-alkynyl, represents optionally cyano-, halogen- or C₁-C₄-alkyl-substituted C₃-C₆-cycloalkyl or C₃-C₆-cycloalkyl-C₁-C₄-alkyl, represents in each case optionally nitro-, cyano-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, phenyl-, phenoxy- or piperazinyl- (which is optionally substituted by C₁-C₄-alkyl or by optionally halogen-, C₁-C₄-allyl- or C₁-C₄-alkoxy-substituted phenyl) substituted C₆-C₁₀-aryl or C₆-C₁₀-aryl-C₁-C₄-alkyl, or represents in each case optionally nitro-, cyano-, halogen-, C₁-C₄-alkyl-, C₁-C₄-haloalkyl-, C₁-C₄-alkoxy- or C₁-C₄-haloalkoxy-substituted heterocyclyl or heterocyclyl-C₁-C₄-alkyl having up to 6 carbon atoms and at least one heteroatom from the group consisting of N, O, S in the heterocyclyl group.

3. A pesticide, characterized in that it comprises at least one compound of the formula (I) as claimed in claim 1 in addition to extenders and/or surfactants.

4. A method for controlling pests, characterized in that compounds of the formula (I) as claimed in claim 1 are allowed to act on pests and/or their habitat.

5. A process for preparing pesticides, characterized in that compounds of the formula (I) as claimed in claim 1 are mixed with extenders and/or surfactants.

6. A compound of the formula (IA) 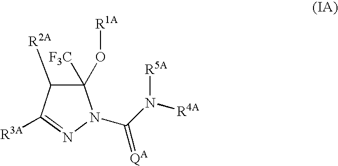
in which
Q^{A} represents oxygen or sulfur,
R^{1A} represents hydrogen or represents in each case optionally substituted alkyl, alkylcarbonyl, cycloalkyl, cycloalkylcarbonyl, aryl, arylcarbonyl, arylalkyl or arylalkylcarbonyl,
R^{2A} represents hydrogen or represents in each case optionally substituted alkyl, alkyl-carbonyl, alkoxycarbonyl, cycloalkyl, aryl, arylalkyl or heterocyclyl,
R^{3A} represents in each case optionally substituted alkyl, cycloalkyl, aryl, arylalkyl or heterocyclyl
R^{2A} and R^{3A} furthermore together represent alkanediyl(alkylene),
R^{4A} represents hydrogen, represents amino, or represents in each case optionally substituted alkyl, alkylcarbonyl, alkylsulfonyl, alkylamino, alkylcarbonylamino, alkylsulfonylamino, alkenyl, alkenylcarbonyl or alkynyl, and
R^{5A} represents in each case optionally substituted alkyl having at least 2 carbon atoms, alkenyl, alkynyl, cycloalkyl, cycloalkylalkyl, aryl, arylalkyl, heterocyclyl or heterocyclylalkyl.

7. The compound of the formula (IA) as claimed in claim 6, in which
Q^{A} represents oxygen or sulfur,
R^{1A} represents hydrogen, represents in each case optionally cyano-, halogen- or C₁-C₄-alkoxy-substituted C₁-C₁₀-alkyl or (C₁-C₁₀-alkyl)carbonyl, represents in each case optionally cyano-, halogen- or C₁-C₄-alkyl-substituted C₃-C₆-cycloalkyl or (C₃-C₆-cycloalkyl)carbonyl, represents in each case optionally nitro-, cyano-, halogen-, C₁-C₄-alkyl-, C₁-C₄-haloalkyl-, C₁-C₄-alkoxy- or C₁-C₄-haloalkoxy-substituted C₆-C₁₀-aryl or (C₆-C₁₀-aryl)carbonyl, or represents in each case optionally nitro-, cyano-, halogen-, C₁-C₄-alkyl-, C₁-C₄-haloalkyl-, C₁-C₄-alkoxy- or C₁-C₄-haloalkoxy-substituted (C₆-C₁₀-aryl)-C₁-C₄-alkyl or (C₆-C₁₀-aryl-C₁-C₄-alkyl)carbonyl,
R^{2A} represents hydrogen, represents optionally cyano-, halogen- or C₁-C₄-alkoxy-substituted C₁-C₁₀-alkyl, (C₁-C₁₀-alkyl)carbonyl or (C₁-C₁₀-alkoxy)carbonyl, represents optionally cyano-, halogen- or C₁-C₄-alkyl-substituted C₃-C₆-cycloalkyl, represents in each case optionally nitro-, cyano-, halogen-, C₁-C₄-alkyl-, C₁-C₄-haloalkyl-, C₁-C₄-alkoxy- or C₁-C₄-haloalkoxy-substituted C₆-C₁₀-aryl or C₆-C₁₀-aryl-C₁-C₄-alkyl, or represents optionally nitro-, cyano-, halogen-, C₁-C₄-alkyl-, C₁-C₄-haloalkyl-, C₁-C₄-alkoxy- or C₁-C₄-haloalkoxy-substituted heterocyclyl having up to 6 carbon atoms and at least one heteroatom from the group consisting of N, O, S,
R^{3A} represents optionally cyano-, halogen- or C₁-C₄-alkoxy-substituted alkyl having from 1 to 10 carbon atoms, represents optionally cyano-, halogen- or C₁-C₄-alkyl-substituted cycloalkyl having from 3 to 6 carbon atoms, represents in each case optionally nitro-, cyano-, halogen-, C₁-C₄-alkyl-, C₁-C₄-haloalkyl-, C₁-C₄-alkoxy- or C₁-C₄-haloalkoxy-substituted aryl or arylalkyl having in each case 6 or 10 carbon atoms in the aryl group and optionally from 1 to 4 carbon atoms in the alkyl moiety, or represents optionally nitro-, cyano-, halogen-, C₁-C₄-alkyl-, C₁-C₄-haloalkyl-, C₁-C₄-alkoxy- or C₁-C₄-haloalkoxy-substituted heterocyclyl having up to 6 carbon atoms and at least one heteroatom from the group consisting of N, O, S,
R^{2A} and R^{3A} furthermore together represent C₃-C₅-alkanediyl(alkylene),
R^{4A} represents hydrogen, represents amino, represents in each case optionally cyano-, halogen- or C₁-C₄-alkoxy-substituted C₁-C₁₀-alkyl, (C₁-C₁₀-alkyl)carbonyl, C₁-C₁₀-alkylsulfonyl, C₁-C₁₀-alkylamino, (C₁-C₁₀-alkyl)carbonylamino or C₁-C₁₀-alkyl-sulfonylamino, or represents in each case optionally halogen-substituted C₂-C₁₀-alkenyl, (C₂-C₁₀-alkenyl)carbonyl or C₂-C₁₀-alkynyl,
R^{5A} represents optionally cyano-, halogen- or C₁-C₄-alkoxy-substituted C₂-C₁₀-alkyl, represents in each case optionally halogen-substituted C₂-C₁₀-alkenyl or C₂-C₁₀-alkynyl, represents optionally cyano-, halogen- or C₁-C₄-alkyl-substituted C₃-C₆-cycloalkyl or C₃-C₆-cycloalkyl-C₁-C₄-alkyl, represents in each case optionally nitro-, cyano-, halogen-, C₁-C₄-alkyl-, C₁-C₄-haloalkyl-, C₁-C₄-alkoxy-, C₁-C₄-haloalkoxy-, C₁-C₄-alkylthio-, C₁-C₄-haloalkylthio-, phenyl-, phenoxy- or piperazinyl- (which is optionally substituted by C₁-C₄-alkyl or by optionally halogen-, C₁-C₄-alkyl- or C₁-C₄-alkoxy-substituted phenyl) substituted C₆-C₁₀-aryl or C₆-C₁₀-aryl-C₁-C₄-alkyl, or represents in each case optionally nitro-, cyano-, halogen-, C₁-C₄-alkyl-, C₁-C₄-haloalkyl-, C₁-C₄-alkoxy- or C₁-C₄-haloalkoxy-substituted heterocyclyl or heterocyclyl-C₁-C₄-alkyl having up to 6 carbon atoms and at least one heteroatom from the group consisting of N, O, S in the heterocyclyl group.

8. A process for preparing compounds of the formula (IA) as claimed in claim 6, characterized in that
(a) 1,3-dicarbonyl compounds of the general formula (II) 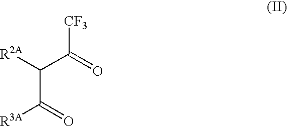
in which R^{2A} and R^{3A} are as defined in claim 6,
are reacted with (thio)semicarbazides of the general formula (III) 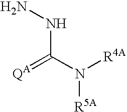
in which Q^{A}, R^{4A} and R^{5A} are as defined in claim 6,
if appropriate in the presence of one or more diluents and if appropriate in the presence of one or more reaction auxiliaries,
or
(b) alkoxyalkenyl ketones of the general formula (IV) 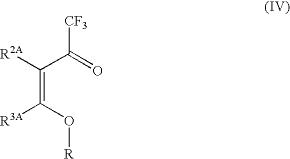
in which
R^{2A} and R^{3A} are as defined in claim 6 and
R represents alkyl
—and where, in addition to the cis- or Z-configuration illustrated by the formula (IV), the corresponding trans- or E-configuration is also meant to be included—are reacted with (thio)semicarbazides of the general formula (III) 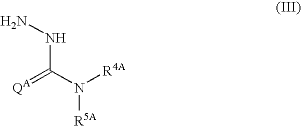
in which Q^{A}, R^{4A} and R^{5A} are as defined in claim 6,
if appropriate in the presence of one or more diluents and if appropriate in the presence of one or more reaction auxiliaries,
or
(c) pyrazolinols of the general formula (V) 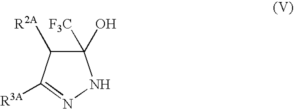
in which R^{2A} and R^{3A} are as defined in claim 6
are reacted with aminocarbonyl compounds of the general formula (VI) 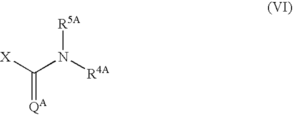
in which
Q^{A}, R^{4A} and R^{5A} are as defined above,
X represents halogen,
or with iso(thio)cyanates of the general formula (VII)
Q^{A}═C═N—R^{5A} (VII)
in which
Q^{A} is as defined in claim 6 and
R^{5A} is as defined above, except for H (hydrogen),
if appropriate in the presence of one or more diluents and if appropriate in the presence of one or more reaction auxiliaries,
and the compounds of the formula (LA) obtained by the processes described under (a), (b) or (c) are, if appropriate, converted by customary methods into other compounds of the formula (I) [or else (IA)].
